(19) United States
(12) Patent Application Publication
(10) Pub. No.: US 2014/0188094 A1
(43) Pub. Date: Jul. 3, 2014

(54) FOCUSED NEAR-INFRARED LASERS FOR NON-INVASIVE VARICOSE VEINS AND OTHER THERMAL COAGULATION OR OCCLUSION PROCEDURES

(71) Applicant: OMNI MEDSCI, INC., Ann Arbor, MI (US)

(72) Inventor: Mohammed N. Islam, Ann Arbor, MI (US)

(73) Assignee: OMNI MEDSCI, INC., Ann Arbor, MI (US)

(21) Appl. No.: 14/108,974

(22) Filed: Dec. 17, 2013

Related U.S. Application Data
(60) Provisional application No. 61/747,492, filed on Dec. 31, 2012.

(51) Int. Cl. A61B 18/20

(52) U.S. Cl. CPC A61B 18/20
USPC 606/7

(57) ABSTRACT

Focused infrared light may be used in a non-invasive varicose vein treatment procedure with infrared light from a plurality of laser diodes that are combined in a multiplexer and coupled to a multi-mode fiber coupled to another fiber or fiber bundle that delivers the light to a lens/mirror assembly for application in the non-invasive procedures. The wavelength of light may be selected near 980 nm, 1210 nm, or 1720 nm to achieve a desired penetration depth and/or for absorption in a particular tissue type or water. Wavelengths near approximately 1100 nm, 1310 nm or 1650 nm may be advantageous for non-invasive procedures through the skin. The light may be focused with lower intensity on the skin or outer tissue to reduce collateral damage and higher intensity at a desired depth to induce thermal coagulation or occlusion at depths of about 1-2 mm or more. Surface cooling techniques, such as cryogenic sprays or contact cooling may be provided.

### CROSS-REFERENCE TO RELATED APPLICATIONS

This application claims the benefit of U.S. provisional application Ser. No. 61/747,492 filed Dec. 31, 2012, the disclosure of which is hereby incorporated in its entirety by reference herein.

This application is related to U.S. provisional application Ser. Nos. 61/747,477 filed Dec. 31, 2012; Ser. No. 61/747,481 filed Dec. 31, 2012; Ser. No. 61/747,485 filed Dec. 31, 2012; Ser. No. 61/747,487 filed Dec. 31, 2012; Ser. No. 61/747,472 filed Dec. 31, 2012; Ser. No. 61/747,553 filed Dec. 31, 2012; and Ser. No. 61/754,698 filed Jan. 21, 2013, the disclosures of which are hereby incorporated in their entirety by reference herein.

This application is being filed concurrently with International Application No. ______ entitled Near-Infrared Lasers For Non-Invasive Monitoring Of Glucose, Ketones, HBA1C, And Other Blood Constituents (Attorney Docket No. OMNI0101PCT); International Application ______ entitled Short-Wave Infrared Super-Continuum Lasers For Early Detection Of Dental Caries (Attorney Docket No. OMNI0102PCT); U.S. application Ser. No. ______ entitled Focused Near-Infrared Lasers For Non-Invasive Vasectomy And Other Thermal Coagulation Or Occlusion Procedures (Attorney Docket No. OMNI0103PUSP); International Application ______entitled Short-Wave Infrared Super-Continuum Lasers For Natural Gas Leak Detection, Exploration, And Other Active Remote Sensing Applications (Attorney Docket No. OMNI0104PCT); U.S. application Ser. No. ______ entitled Short-Wave Infrared Super-Continuum Lasers For Detecting Counterfeit Or Illicit Drugs And Pharmaceutical Process Control (Attorney Docket No. OMNI0105PUSP); and U.S. application Ser. No. ______ entitled Near-Infrared Super-Continuum Lasers For Early Detection Of Breast And Other Cancers (Attorney Docket No. OMNI0107PUSP), the disclosures of which are hereby incorporated in their entirety by reference herein.

### TECHNICAL FIELD

This disclosure relates to lasers and light sources for healthcare, medical, or bio-technology applications including systems and methods for using focused near-infrared light sources for non-invasive varicose vein occlusion and other thermal coagulation or occlusion procedures.

### BACKGROUND AND SUMMARY

Varicose veins are very common in both women and men, and varicose veins may be painful and unattractive. For example, it has been estimated that 41% of women and 15% of men are affected by asymptomatic and visible veins on the legs. Consequently, leg vein therapy is one of the most commonly requested cosmetic procedures. Although these veins may start by being of cosmetic importance, more than half may become symptomatic, particularly if left untreated.

Varicose veins are veins that may have become enlarged and tortuous, and the term commonly refers to veins on the leg. Varicose veins are most common in the superficial veins of the legs, which are subject to high pressure when standing. Superficial vein is a term used to describe a vein that is close to the surface of the body. The term is used to differentiate veins that are close to the surface from veins that are far from the surface, which are known as deep veins. Because most of the blood in the legs is returned by the deep veins, the superficial veins, which return only about 10% of the total blood of the legs, can usually be removed or ablated without serious harm.

The heart pumps oxygen-rich blood into a large artery known as the aorta. The aorta divides into two main arteries, which continue to branch into smaller arteries delivering blood to the rest of the body. Once the oxygen has been delivered, veins carry the blood back to the heart. However, unlike arteries the veins are dependent on one-way valves to keep blood moving in an upward motion. The muscles of the legs help push the blood through the veins, while the one-way valves close and prevent the blood from falling back towards the feet. When the one-way valves fail to close properly, blood may reverse its flow. This may cause increased pressure in the veins, and over time may cause them to swell and become bulging, varicose veins.

Depending on the severity of the varicose veins, treatments may include non-surgical as well as surgical procedures. Non-surgical treatments include sclerotheraphy, elastic stockings, elevating the legs, and exercise. In sclerotheraphy procedures, a medicine may be injected into the blood vessel, causing the vessel to shrink. The traditional surgical treatment has been vein stripping to remove the affected veins. Alternative techniques are available as well, such as ultrasound-guided foam sclerotherapy, radiofrequency ablation, and endovenous laser treatment (ELT). ELT is a relatively newer, minimally invasive treatment for varicose veins. ELT uses a laser that has been fit with a laser fiber tip that is used to introduce light energy into the vein to be treated. The light energy, in turn, may damage the inner vein wall, causing the collagen fibers to contract and the vein to collapse.

As an alternative to ELT, it would be desirable to have a non-invasive treatment or method for treating varicose veins. By using appropriate wavelengths of infrared light, the penetration depth may be large enough to reach non-invasively the varicose veins. The skin may comprise water, collagen, adipose and elastin, so larger penetration depth may be achieved by avoiding absorption peaks in these constituents with appropriate selection of the infrared wavelengths. Also, by focusing the light, the intensity of the light may be lower on the skin surface and higher at the vein vessel wall and lumen, thus permitting less damage to the skin while heating the vessel. In addition, surface cooling may be used to prevent damage to the top layer of the skin by limiting the temperature rise at the skin. Hence, by using a combination of cooling and/or focused light and infrared light, it may be possible to non-invasively cause occlusion of the varicose veins without substantially damaging the skin, or at least the top layer of the skin. Moreover, this technique may be beneficially applied to other procedures such as treatment of finger or toe nails from fungal infection, treatment of hemorrhoids, laser tissue welding, dermatology treatments including treatment for acne or sebaceous hyperplasia, and non-invasive vasectomy procedures.

In one embodiment, a therapeutic system includes a light source generating an output optical beam comprising a plurality of semiconductor sources generating an input optical beam, a multiplexer configured to receive at least a portion of the input optical beam and to form an intermediate optical beam, and one or more fibers configured to receive at least a portion of the intermediate optical beam and to form the output optical beam, wherein the output optical beam comprises one or more optical wavelengths, and wherein at least a portion of the one of more fibers is a fused silica fiber with a core diameter less than approximately 400 microns. An interface device is configured to receive a received portion of the output optical beam and to deliver a delivered portion of the output optical beam to a sample, wherein the interface device comprises one or more lenses to focus at least a part of the delivered portion of the output optical beam on the sample, and wherein the interface device further comprises a surface cooling apparatus to reduce damage to a top surface of the sample. The part of the delivered portion of the output optical beam is at least partially absorbed in the sample to thermally damage at least a part of the sample, and a sample temperature in the part of the sample reaches about 65 Celsius or higher, while a cover temperature at the top surface of the sample remains less than about 65 Celsius. The output optical beam comprises a fluence less than about 250 Joules per centimeter squared.

In another embodiment, a therapeutic system includes a light source generating an output optical beam comprising one or more semiconductor sources generating an input optical beam, one or more fibers configured to receive at least a portion of the input optical beam and to form an intermediate optical beam, and a light guide configured to receive at least a portion of the intermediate optical beam and to form the output optical beam, wherein the output optical beam comprises one or more optical wavelengths. An interface device is configured to receive a received portion of the output optical beam and to deliver a delivered portion of the output optical beam to a sample, wherein the interface device comprises one or more lenses to focus at least a part of the delivered portion of the output optical beam on the sample, and wherein the interface device further comprises a surface cooling apparatus to reduce damage to a top surface of the sample. At least some of the part of the delivered portion of the output optical beam is at least partially absorbed in the sample to thermally damage at least a part of the sample, and a sample temperature in the part of the sample reaches about 65 Celsius or higher, while a cover temperature at the top surface of the sample remains less than about 65 Celsius.

In yet another embodiment, a method of therapy includes generating an output optical beam comprising generating an input optical beam from one or more semiconductor sources, forming an intermediate optical beam after propagating at least a portion of the input optical beam through one or more fibers, and guiding at least a portion of the intermediate optical beam and forming the output optical beam, wherein the output optical beam comprises one or more optical wavelengths. The method may also include receiving a received portion of the output optical beam and delivering a delivered portion of the output optical beam to a sample, focusing at least a part of the delivered portion of the output optical beam on the sample, and cooling a top surface of the sample. The method may further include absorbing at least some of the part of the delivered portion of the output optical beam in the sample, damaging thermally at least a part of the sample, and wherein a sample temperature in the part of the sample reaches about 65 Celsius or higher, while a cover temperature at the top surface of the sample remains less than about 65 Celsius.

### BRIEF DESCRIPTION OF THE DRAWINGS

For a more complete understanding of the present disclosure, and for further features and advantages thereof, reference is now made to the following description taken in conjunction with the accompanying drawings, in which:

FIG. 1A illustrates the vein anatomy in a typical human leg.

FIG. 1B shows a cross-sectional view of the veins below the skin.

FIG. 2 exemplifies the progression of incompetent valves.

FIG. 3 illustrates more details of the superficial vein system and the deep vein system.

FIG. 4A shows one embodiment of the endovenous laser treatment procedure.

FIG. 4B shows exemplary the effect on the vein of endovenous laser treatment.

FIG. 5 illustrates the overlap of the absorption coefficients for water and tissue scattering, adipose, collagen and elastin; vertical lines are also drawn to highlight the wavelengths near 1210 nm and 1720 nm; the adipose and water absorption coefficients as well as the scattering loss are shown on a calibrated scale, while the collagen and elastin are in arbitrary units.

FIG. 6 shows one exemplary set-up for non-invasive treatment of varicose veins.

FIG. 7 illustrates one embodiment of the light input to the non-invasive varicose vein treatment assembly. The light source may comprise, for example, LED's, laser diodes, fiber lasers, or super-continuum lasers.

FIG. 8 shows one embodiment of the non-invasive varicose vein treatment apparatus that may have a focused laser beam and optional cryogenic cooling spray.

FIG. 9 illustrates another embodiment of the non-invasive varicose vein treatment apparatus that may have a focused laser beam and optional surface cooling by flowing fluid in close proximity to the skin.

FIG. 10 shows yet another embodiment of the non-invasive varicose vein treatment apparatus that may comprise multiple collimated or focused light beams and optional surface cooling.

FIG. 11 illustrates in the top right corner a schematic of the toenail anatomy. The bottom left includes the absorption coefficient for water-containing keratin and keratin approximately alone. The bottom right shows the attenuation coefficient (absorption plus scattering) of seven nail samples that were allowed to stand in a humidity level of 14%.

FIG. 12 shows an experimental set-up for testing chicken breast samples using collimated light. In this experiment, the collimated light has a beam diameter of about 3 mm.

FIG. 13 plots the measured depth of damage (in millimeters) versus the time-averaged incident power (in Watts). Data is presented for laser wavelengths near 980 nm, 1210 nm and 1700 nm, and lines are drawn corresponding to penetration depths of approximately 2 mm, 3 mm, and 4 mm.

FIG. 14 illustrates the optical absorption or density as a function of wavelength between approximately 700 nm and 1300 nm for water, hemoglobin and oxygenated hemoglobin.

FIG. 15 shows a set-up used for in vitro damage experiments using focused infrared light. After a lens system, the tissue is placed between two microscope slides.

FIG. 16 presents histology of renal arteries comprising endothelium, media and adventitia layers and some renal nerves in or below the adventitia. (A) No laser exposure. (B) After focused laser exposure, with the laser light near 1708 nm.

FIG. 17 illustrates the experimental set-up for ex vivo skin laser treatment with surface cooling to protect the epidermis and top layer of the dermis.

FIG. 18 shows MTT histo-chemistry of ex vivo human skin treated with ˜1708 nm laser and cold window (5 seconds precool; 2 mm diameter spot exposure for 3 seconds) at 725 mW (A and B) corresponding to ˜70 J/cm² average fluence and 830 mW (C and D) corresponding to ˜80 J/cm² average fluence.

FIG. 19 illustrates a block diagram or building blocks for constructing high power laser diode assemblies.

FIG. 20 shows a platform architecture for different wavelength ranges for an all-fiber-integrated, high powered, super-continuum light source.

FIG. 21 illustrates one embodiment for a short-wave infrared super-continuum light source.

FIG. 22 shows the output spectrum from the SWIR SC laser of FIG. 21 when about 10 m length of fiber for SC generation is used. This fiber is a single-mode, non-dispersion shifted fiber that is optimized for operation near 1550 nm.

FIG. 23 illustrates high power SWIR-SC lasers that may generate light between approximately 1.4-1.8 microns (top) or approximately 2-2.5 microns (bottom).

FIG. 24A illustrates a block diagram of one embodiment of an infrared fiber laser operating near 1720 nm;

FIG. 24B shows details of one specific example of an infrared fiber laser operating at approximately 1708 nm; the top part of the figure illustrates one embodiment of the pump fiber laser, and the bottom part of the figure illustrates one embodiment of the cascaded Raman oscillator or cascaded Raman wavelength shifter;

FIG. 25A illustrates a block diagram yet another embodiment of an infrared fiber laser operating near 1210 nm;

FIG. 25B shows details of one specific example of an infrared fiber laser operating at approximately 1212 nm; the top part of the figure illustrates one embodiment of the pump fiber laser, and the bottom part of the figure illustrates one embodiment of the cascaded Raman oscillator or cascaded Raman wavelength shifter.

### DETAILED DESCRIPTION OF EXAMPLE EMBODIMENTS

As required, detailed embodiments of the present disclosure are described herein; however, it is to be understood that the disclosed embodiments are merely exemplary of the disclosure that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present disclosure.

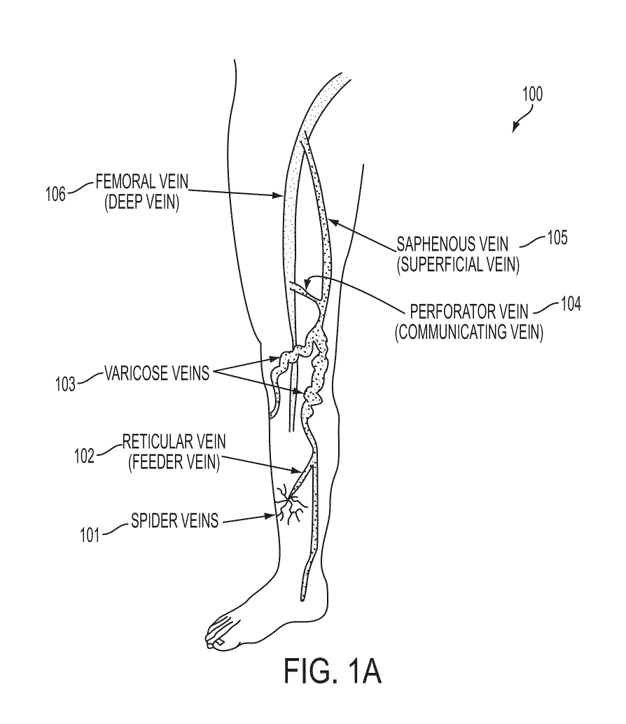

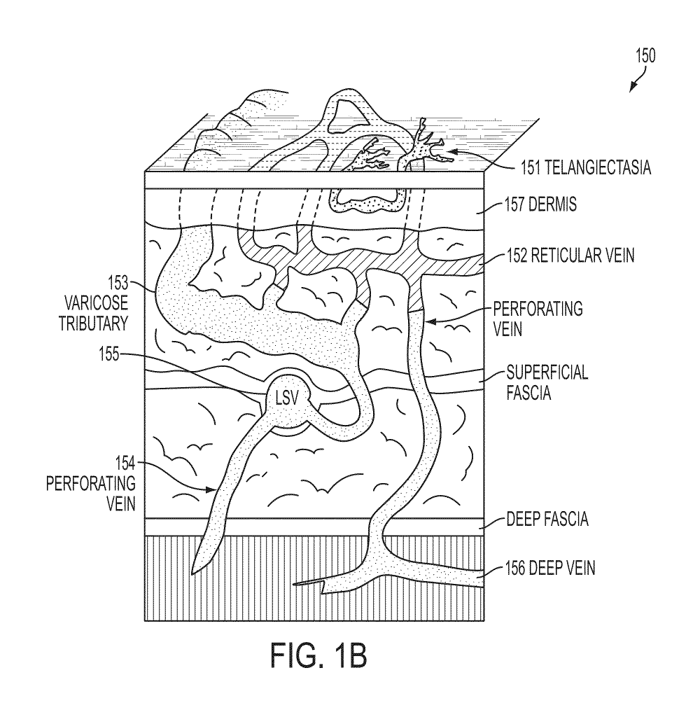

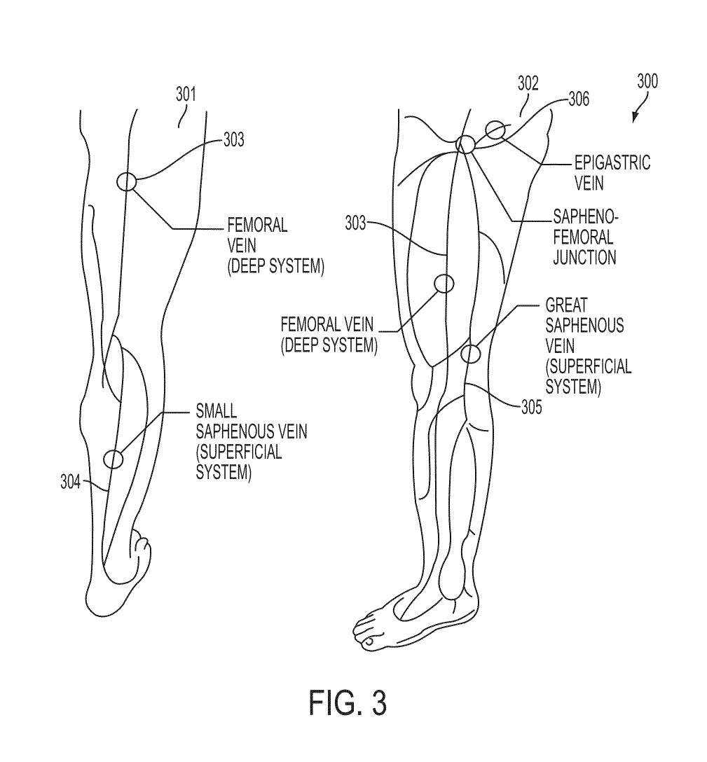

The vein anatomy in a typical human leg is illustrated in FIG. 1. For example, FIG. 1A shows the veins in the leg 100, while FIG. 1B shows a cross-sectional view of the veins below the skin 150. The telangiectasia or spider veins 101,151 are typically small veins near the surface of the skin (typically less than approximately 1 mm in size). The reticular veins or feeder veins 102,152 are slightly deeper below the dermis 157 and typically range in size between approximately 1-3 mm. The varicose veins 103,153 are yet larger veins that may be typically larger than 3 mm in size. The varicose veins 103,153 may extend from the saphenous vein 105, 155 (see also FIG. 3). The saphenous veins 105,155 are parts of the superficial vein system. Also, the saphenous veins 105,155 may be coupled to the femoral vein 106,156, which is part of the deep vein system. Moreover, there may be perforator veins 104,154, which may be communicating veins between the superficial veins 105,155 and the deep veins 106,156.

Varicose veins may lead to unsightly bluish-purple blemishes, but they may also cause discomfort and disability. Varicose veins are abnormally dilated, tortuous, superficial veins caused by incompetent venous valves. Most commonly, the varicose vein condition may affect the lower extremities, such as the saphenous veins. Varicose vein difficulty may be due to valve defects inside the veins themselves. In particular, they may occur when the stop valves within the veins fail to propel oxygen depleted blood back to the heart from the legs. Because of the valve malfunction, blood may be allowed to pool in the leg veins, causing them to become distorted and painful and, if left untreated, may lead to ulceration and skin damage.

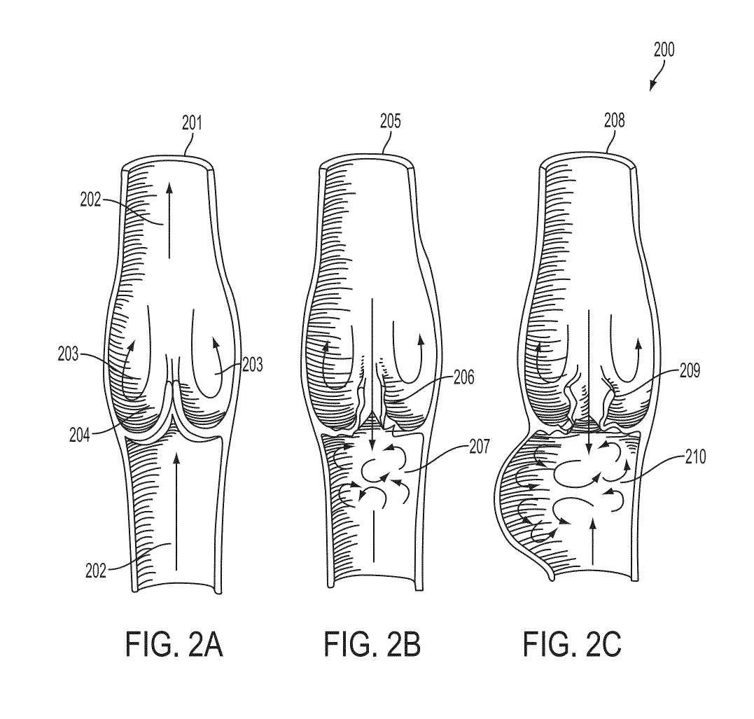

As an example, FIG. 2 shows the incompetent valve progression 200. In the figure, gravity pulls the blood down 203, and blood pressure pulls the blood upwards 202. In FIG. 2A 201, a proper valve 204 permits the blood to flow upward 202 due to blood pressure, but the valve prevents the gravity-pulled blood 203 from passing the valve 204. FIG. 2B 205 shows that when the valve becomes at least partially incompetent 206, at least some of the gravity-pulled blood may pass the valve 206 and permit blood build-up 207 below the valve. As the incompetent valve progresses 208, in FIG. 2C is shown that a blood build-up 210 may occur below the valve 209, leading to the formation of structures such as varicose veins.

FIG. 3 illustrates more details 300 of the superficial vein system and the deep vein system. In particular, the left of the figure shows the back view of the leg 301, while the right of the figure shows the front view of the leg 302. The femoral vein 303 is part of the deep system. Along the back of the leg is the small (or lesser) saphenous vein 304, which is part of the superficial system. Running along the length of the leg is the great (or large) saphenous vein 305, which is also a part of the superficial system. The great saphenous vein 305 may join the femoral vein 303 at the sapheno-femoral junction 306.

One example of a laser-based treatment for varicose veins is endovenous laser treatment, or ELT. ELT may be a minimally invasive, ultrasound-guided technique used for treating varicose veins using laser energy commonly performed by an interventional radiologist or vascular surgeon. In ELT, an optical fiber may be inserted into the vein to be treated, and laser light, normally in the infrared portion of the spectrum, is shone into the interior of the vein. This may cause the vein to contract, and the optical fiber is slowly withdrawn.

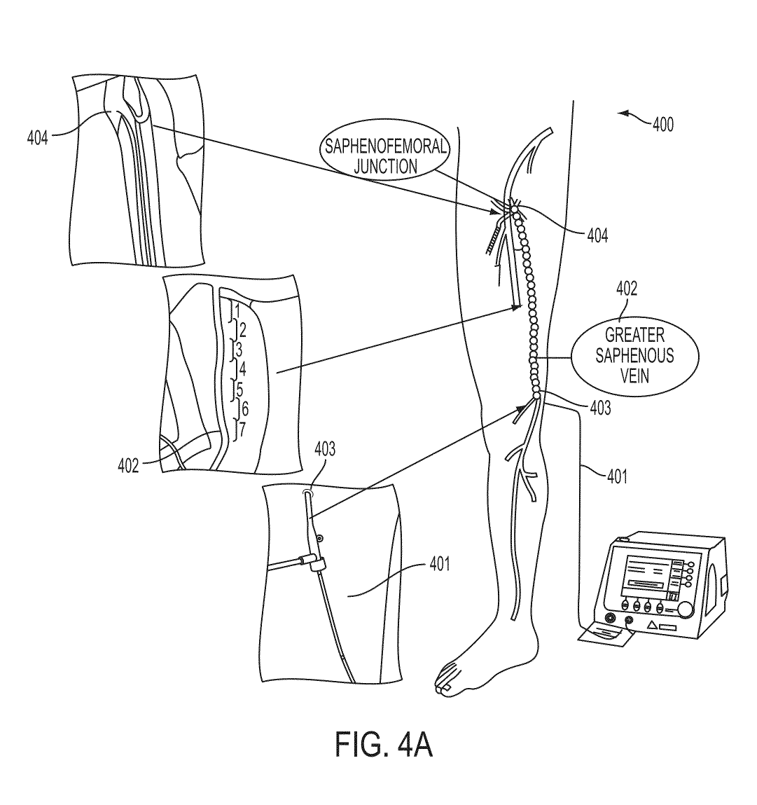

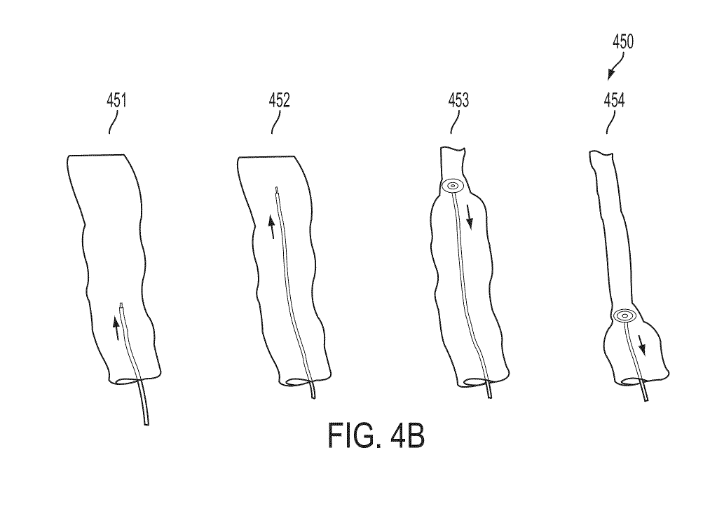

One embodiment of the ELT procedure 400 is shown in FIG. 4A. During the procedure, a catheter bearing a laser fiber 401 may be inserted under ultrasound guidance into the great saphenous vein 402 (alternately, not shown, it may be inserted into the small saphenous vein) through a small puncture 403. The catheter may then be advanced (also under ultrasound guidance) to the sapheno-femoral junction 404 near the groin (alternately, not shown, it may be inserted up to the knee crease for treating the small saphenous vein). In addition, dilute local anesthesia may be injected around and along the vein. For example, a treatment area may be anesthetized with a common skin anesthetic, lidocaine. Following the treatment, the patient may be required to wear a compression stocking for a period of time.

The principle of ELT may be ablation and photocoagulation of the vein interior by laser-induced thermal effects. In one embodiment, the effect on the vein 450 is illustrated in FIG. 4B. The fiber and catheter are first inserted, such as in 451,452. Then, laser energy may be delivered through the fiber as the fiber and catheter assembly is withdrawn 453,454. During fiber withdrawal the vein wall may be occluded, perhaps even irreversibly damaged. Some of the parameters governing the laser treatment include the laser source wavelength, pulse form and power, and the optical fiber withdrawal rate. In one embodiment, the withdrawal rate of the fiber affects the amount of energy delivered to the vein wall. However, the diameter of the vein changes along the length of the vein, from approximately 10-20 mm near the sapheno-femoral junction to about 2-3 mm at the entry point. Thus, if it is desired to deliver approximately constant energy per vein wall surface area, then either the withdrawal rate and/or the laser energy should be varied during the procedure.

Some of the prior techniques to treat varicose veins have attempted to heat the vessel by targeting the hemoglobin in the blood, and then having the heat transfer to the vessel wall. For these embodiments, lasers emitting wavelengths of approximately 500 nm to 1100 nm have been used. Attempts have also been made to optimize the laser energy absorption by utilizing local absorption peaks of hemoglobin at 810 nm, 940 nm, 980 nm and 1064 nm. However, the heat transfer method used in these instances may result in poor efficiency in heating the collagen in the vessel wall and damaging or destroying the endothelial cells. In some embodiments, it may also be desired to limit the heating to approximately 80-85 degrees Celsius to avoid boiling, vaporization and carbonization of tissues. In addition, blood may coagulate at about 80 degrees Celsius. Regions of blood that have coagulated and remain in the vein may prevent the vein from completely collapsing on itself. Moreover, heating the endothelial wall to 85 degrees Celsius may result in heating the vein media to approximately 65 degrees Celsius, which is known to lead to collagen contraction or shrinkage.

In various embodiments, methods to treat varicose veins by targeting the vessel wall directly with a more appropriate wavelength of laser light have been contemplated. In particular, laser wavelengths may be employed that transmit through any residual blood in the vessels, yet the laser light may be absorbed by the water and collagen of the vessel wall. For example, experiments have demonstrated that laser energy may be absorbed directly in the vessel walls using wavelengths in the range of approximately 1200 nm to 1800 nm. One advantage of this range of wavelengths is that energy may be absorbed more uniformly with less risk of hot spots, boiling, or explosions caused by blood pockets. Also, this wavelength range may lead to less pain and collateral bruising, perhaps because very little light transmits outside the vessel to cause damage.

### Non-Invasive Laser Treatment from Above the Skin

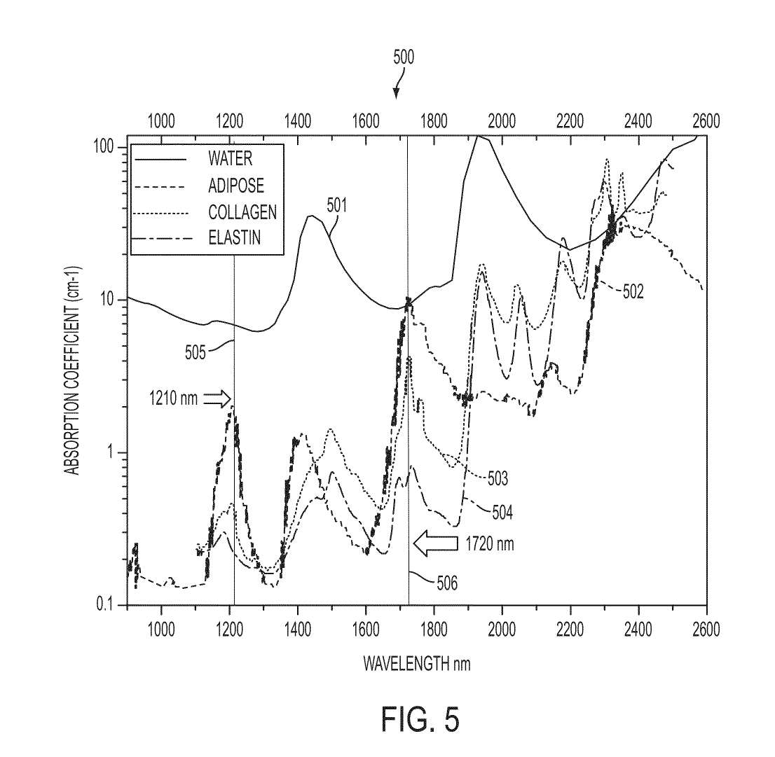

Since in a non-invasive varicose vein treatment technique the light would have to transmit through the dermis 157, the absorption coefficient for the various skin constituents should be examined. One other consideration may be the scattering through tissue in the dermis. Although the absorption coefficient may be useful for determining the material in which light of a certain infrared wavelength will be absorbed, to determine the penetration depth of the light of a certain wavelength may also require the addition of scattering loss to the curves. In FIG. 5 shown are 500 the absorption coefficients for water (with scattering) 501, adipose 502, collagen 503 and elastin 504. Note that the absorption curves for water 501 and adipose 502 are calibrated, whereas the absorption curves for collagen 503 and elastin 504 are in arbitrary units. Also shown are vertical lines demarcating the wavelengths near 1210 nm 505 and 1720 nm 506. The water curve 501 includes the scattering loss curve in addition to the water absorption, and it may be noted that the scattering loss can be significantly higher at shorter wavelengths. In general, the water absorption increases with increasing wavelength. With the increasing absorption beyond about 2000 nm, it may be difficult to achieve deeper penetration into biological tissue in the infrared wavelengths beyond approximately 2500 nm.

In one embodiment, the vein or vessel walls may be modeled as smooth muscle tissue. As an example, smooth muscle tissue or tunica media may comprise protein, which may have an absorption coefficient similar to collagen (e.g., 503). Hence, by selecting wavelengths near peaks of absorption for collagen 503 in FIG. 5, the absorption for the light in the vessel wall may be higher. In one embodiment, wavelengths near approximately 1210 nm or 1720 nm may permit absorption in the vessel wall as well as reasonable transmission through the dermis. However, these wavelengths also correspond to apeak in adipose tissue absorption 502, which may make it difficult to reach the veins through the subcutaneous fat.

In one embodiment, one desired goal for a non-invasive varicose vein treatment procedure is to cause coagulation (probably through a thermal process) or occlusion of the vein or vessel wall with minimal damage to the skin above. From FIG. 1B, this corresponds to leaving undamaged the top several millimeters or more of skin (epidermis, dermis, and some of the subcutaneous fat above the varicose veins). Light can be used to perform the procedure, where the thermal coagulation may occur through heat generated by absorption of the light in the vein lumen and vein wall. However, the wavelength of light should be selected appropriately to achieve a non-invasive procedure. For example, the light should be able to penetrate deep enough to reach through the dermis and subcutaneous fat layers to reach varicose veins. For example, the penetration depth may be defined as the inverse of the absorption coefficient, although it may also be necessary to include the scattering for the calculation. More generally, the light penetration should be deep enough to permit adequate light intensity at the vein (wall and lumen) to cause thermal coagulation or occlusion. Second, to generate the heat for coagulation, the light should be at least partially absorbed in the vein lumen (which may be modeled as water) and perhaps also some absorption in the vessel wall (tissue also has a significant water content).

In a particular embodiment, wavelengths for the non-invasive procedure may be selected based on the absorption curves 500 in FIG. 5. The dermis may have a significant amount of water 501, as well as some collagen 503, elastin 504 and adipose 502. The subcutaneous fat layer below the skin may have significant adipose 502 content. Therefore, to achieve penetration deep enough to reach the varicose veins, wavelengths may correspond to local minima in water 501 and adipose 502 absorption, as well as potentially local minima in collagen 503 and elastin 504 absorption. For example, wavelengths near approximately 1100 nm, 1310 nm, or 1650 nm may be advantageous for non-invasive procedures. More generally, wavelength ranges of approximately 900 nm to 1150 nm, 1280 nm to 1340 nm, or 1550 nm to 1680 nm may be advantageous for non-invasive procedures.

A light based procedure may also be aided by several means of preserving the top layers of the skin. In one embodiment, the light could be focused to a depth of approximately the varicose veins (e.g., ultrasound imaging may be used to locate the varicose veins). By focusing the light, a funnel may be created for the light intensity, with a lower intensity on the epidermis and dermis layers and higher intensity at the varicose veins. In another embodiment, surface cooling may be added to preserve the epidermis and at least a fraction of the dermis. For example, surface cooling may be a common technique used in laser based dermatology and cosmetic surgery applications. Surface cooling methods may include a cryo-spray, air cooling, or a water/liquid cooled surface in contact with skin. The water/liquid cooled surface may be in contact surrounding the laser beam spot, or the laser beam may transmit through the surface if it is at least partially transmitting at the laser wavelength. Although two techniques for preserving the skin have been described, combinations of the two or other techniques may also be used and are intended to be covered by this disclosure.

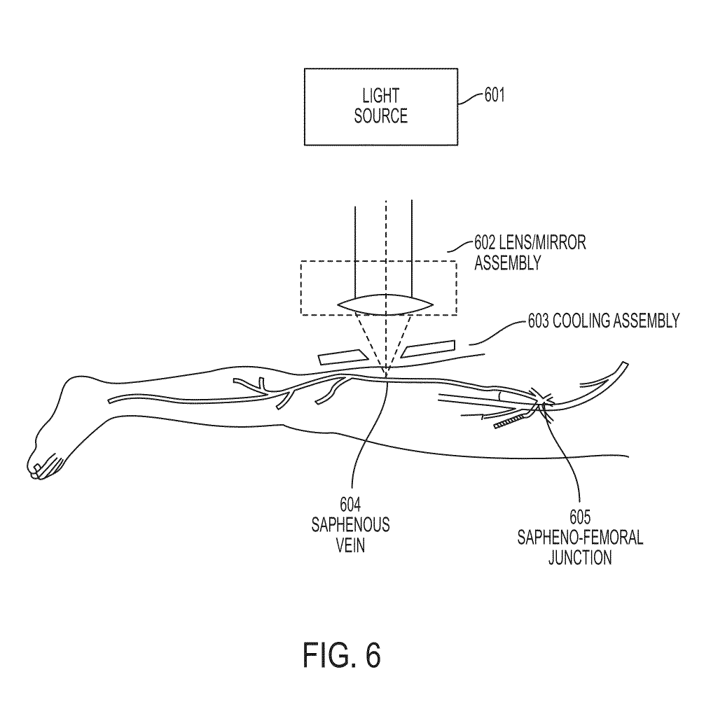

An exemplary set-up for non-invasive varicose vein treatment 600 is illustrated in FIG. 6. The light source 601 may provide sufficient power levels of light in some of the wavelength bands near approximately 1100 nm, 1310 nm, or 1650 nm, although other wavelengths could be used as well. This light may be directed or communicated to a lens and/or mirror assembly 602, which may focus the light to the depth approximately of the saphenous vein 604. In one embodiment, it may be advantageous to have an adjustable focal length system 602, so that the light may follow the depth of the varicose vein 604. In addition, there may be a cooling assembly 603 placed around the spot where the laser light impinges on the skin. In a particular embodiment, the lens and/or mirror assembly 602 and the cooling assembly 603 may be mounted in a hand-held device, which can then be positioned and moved along the desired section of the saphenous vein 604, perhaps even reaching up to the sapheno-femoral junction 605. This is just one embodiment of the non-invasive device, but other configurations may also be used within the scope of this disclosure.

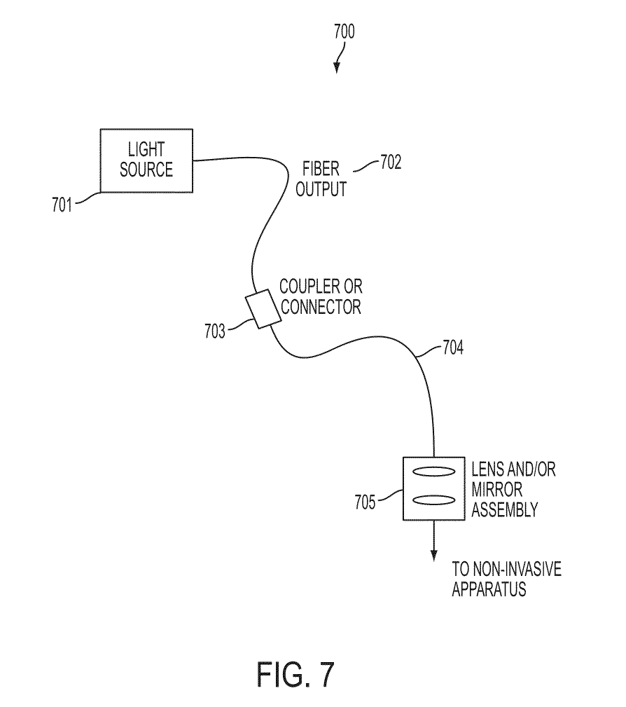

In one embodiment, the light input 700 to the non-invasive varicose vein treatment assembly may be as shown in FIG. 7. The light source 701 may be one or more laser diodes, a fiber laser, or perhaps even a lamp or LED's (light sources are described in greater detail herein). The light source output may be delivered through a light pipe 702, which may be one or more single mode or multi-mode fibers. In a particular embodiment, the light source output pipe 702 may be attached to a coupler or connector 703. In turn, the light pipe or fiber optic or fiber bundle 704 may be coupled to the connector 703, and the light may then be delivered to a lens and/or mirror assembly 705 coupled to the non-invasive apparatus. Although one example is shown in FIG. 7, various components may be added or removed from the light source assembly 700, and these variations are intended to be covered by this disclosure.

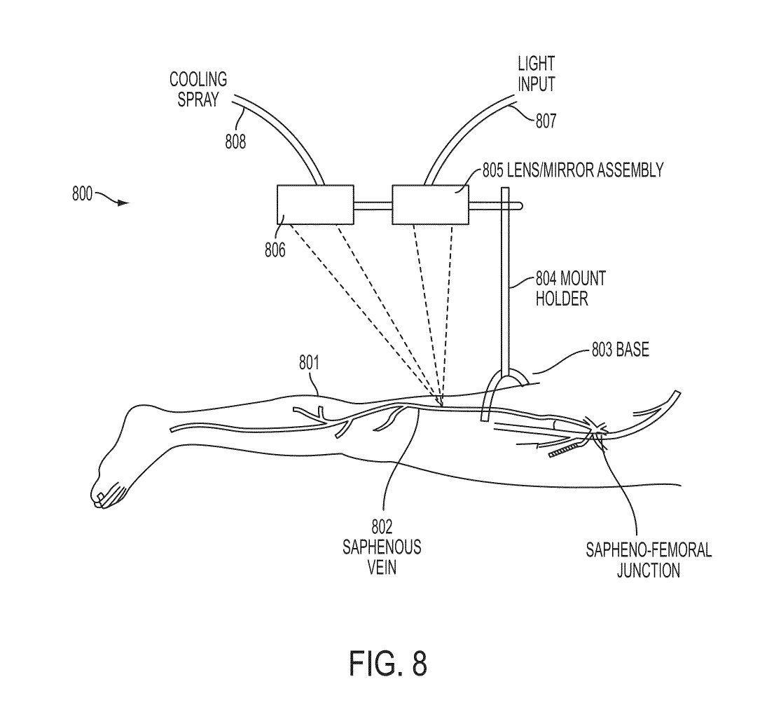

Another embodiment of the non-invasive apparatus 800 is illustrated in FIG. 8. In particular, this embodiment contemplates a focused laser beam assembly 805 with an optional cryogenic cooling spray attachment 806. A mount or assembly 804 may be used to secure the light input and cooling spray input relative to the leg with some sort of base 803. Attached to the mount 804 may be a lens and/or mirror assembly 805 that may receive the light input 807 from an apparatus such as in FIG. 7 and then collimate or focus the light onto the skin 801 and/or the saphenous vein 802. The head for the cooling spray 806 may also be attached to the mount 804, and the head 806 may receive a cooling spray 808 from an external unit. As one particular embodiment, the cooling spray 806 and/or 808 may be a dynamic cooling device made by Candela Laser. The spray 806 may cool the area near and surrounding where the laser beam is incident on the skin 801. Although one embodiment is shown in FIG. 8, some of the parts may be removed or other parts may be added, and these variations are also intended to be covered by this disclosure.

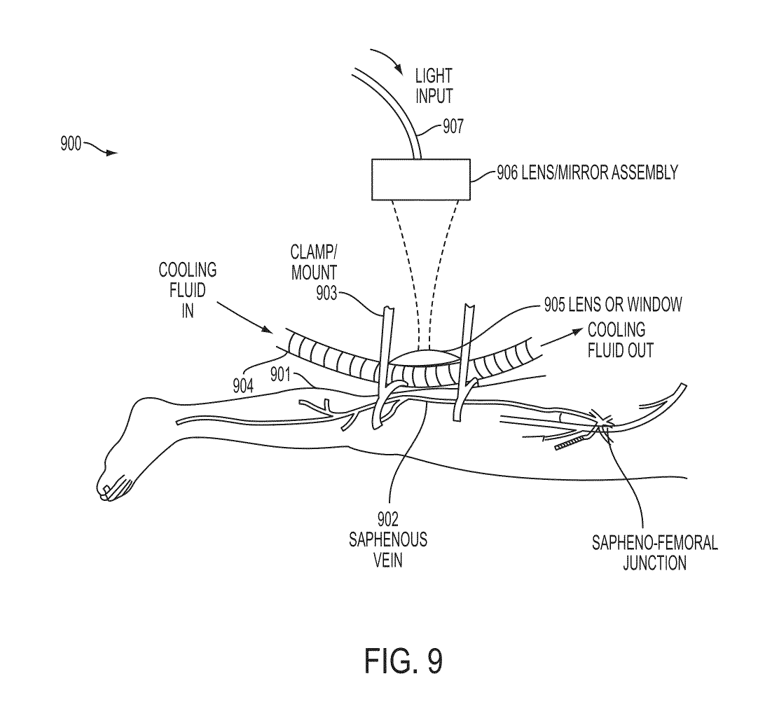

Another embodiment of the non-invasive apparatus 900 is illustrated in FIG. 9. In this embodiment, optionally a surface cooling apparatus 904 may be used, where a cooling fluid may be flowed either touching or in close proximity to the skin 901. In this particular embodiment, a cylindrical region may be exposed by the light, where the cylindrical length may be several millimeters in length and defined by a clamp or mount 903 that may rest on the leg segment being treated. In this example, a window 905 is also shown on the cylindrical surface for permitting the light to be incident on the skin 901 and varicose vein 902, and the window 905 may also be a lens. For instance, if a round spot is desired, then a circular or spherical lens window 905 may be used. On the other hand, if a line is desired, then a cylindrical lens window 905 may be used. One advantage of placing a lens 905 in close proximity to the skin 901 and varicose vein 902 may be that a high numerical aperture, NA, lens may be used, so the cone angle of the light can be relatively steep. A high NA lens may help to increase the difference in light intensity between the skin 901 and the varicose vein 902. The light input 907 may be received from a light source as shown in FIG. 7. A lens and/or mirror assembly 906 may be used to couple the light input 907 to the lens or window 905, either directly or indirectly. The lens and/or mirror assembly 906 may also be coupled to a positioning clamp or mount assembly 903. Although one embodiment is shown in FIG. 9, some of the parts may be removed or other parts may be added, and these variations are also intended to be covered by this disclosure.

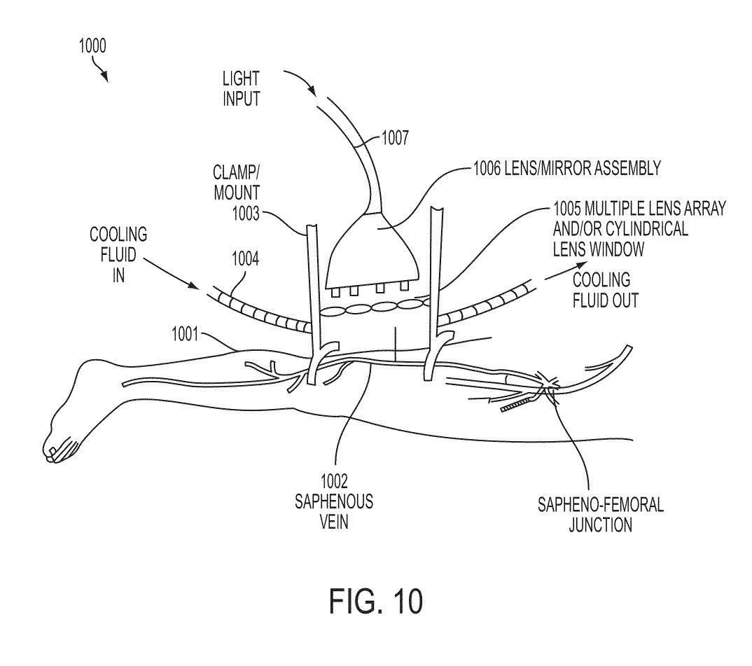

In some instances it may be desirable to create multiple locations of focused light on the varicose vein. For example, the speed of the treatment may be increased by causing thermal coagulation or occlusion at multiple locations One way to accomplish this may be to slide the assemblies and/or the light source such as shown in FIG. 6, 8 or 9 along the length of the varicose vein. In yet another embodiment shown in FIG. 10, multiple collimated or focused light beams may be created in one assembly 1000. In this embodiment, optionally a surface cooling apparatus 1004 may be used, where a cooling fluid may be flowed either touching or in close proximity to the skin 1001. Also, in this particular embodiment a cylindrical assembly may optionally be used, where the cylindrical length may be several millimeters in length and defined by a clamp or mount 1003 placed on or near the leg. The light input 1007 may be received from a light source as shown in FIG. 7, which may use a fiber or fiber bundles to couple the light to the lens/mirror assembly 1006. A lens and/or mirror assembly 1006 may be used to couple the light input 1007 to the lenslet array or window 1005, either directly or indirectly. The lens and/or mirror assembly 1006 may also be coupled to the clamp or mount assembly 1003.

In the embodiment of FIG. 10, a window and/or lenslet array 1005 is also shown on the cylindrical surface for permitting the light to be incident on the skin 1001 and varicose vein 1002 at multiple spots. The lenslet array 1005 may comprise circular, spherical or cylindrical lenses, depending on the type of spots desired. As before, one advantage of placing the lenslet array 1005 in close proximity to the skin 1001 and varicose vein 1002 may be that a high NA, lens may be used. Also, the input from the lens and/or mirror assembly to the lenslet array 1005 may be single large beam, or a plurality of smaller beams. In one embodiment, a plurality of spots may be created by the lenslet array 1005 to cause a plurality of locations of thermal coagulation in the varicose vein 1002. Although four spots are shown in FIG. 10, any number of spots may be used and are intended to be covered by this disclosure.

Although several embodiments of non-invasive varicose vein apparatuses are illustrated in FIGS. 6-10, some of the parts may be removed or other parts may be added, and these variations are also intended to be covered by this disclosure. Also, different combinations of these techniques may be employed, and other techniques may also be used and are intended to be covered by this disclosure. For example, in some instances only focused light may be used, in other instances only surface cooling or cryogenic sprays may be used, and in yet other embodiments a combination of the two may be used. Moreover, the clamps, mounts and holders are shown in simple design for illustrative purposes, but human factors engineering may be used to make these more user friendly or ergonomic design. These and other variations are also intended to be covered by this disclosure.

The lens and/or mirror assemblies may comprise one or more lenses, microscope objectives, curved or flat mirrors, lens tipped fibers, or some combination of these elements. As an example, the optics such as used in a camera may be employed in this arrangement, provided that the optics is substantially transparent at the light wavelengths being used. Moreover, reflections and losses through the optics may be reduced by applying anti-reflection coatings, and chromatic dispersion may be reduced by using reflective optics rather than refractive optics. Although a particular method of focusing the light has been described, other methods may also be used and are intended to be covered by this disclosure.

### Other Applications of Focused Infrared Light

Described herein are just some examples of the beneficial use of infrared laser treatment based on using focused light and/or surface cooling. However, many other medical procedures can use the infrared light consistent with this disclosure and are intended to be covered by the disclosure. For example, although non-invasive varicose vein treatment has been described in detail as one embodiment, more generally the focused infrared light may be used to thermally coagulate or occlude relatively shallow vessels non-invasively or minimally invasively while preserving or minimizing damage to the top layer of the skin or tissue. Other applications where this more general technique may be beneficial include treatment of finger or toe nails from fungal infection, treatment of hemorrhoids, laser tissue welding, dermatology treatments including treatment for acne or sebaceous hyperplasia, and non-invasive vasectomy procedures, for example.

In one embodiment, it may be advantageous to use focused infrared light for treatment of finger or toe nail fungus. Onychomycosis, or fungal infection of nails (most often on the toes) affects about 12% of Americans, according to the American Academy of Dermatology. Toenail laser treatment may offer an attractive alternative to oral medication, which carries a risk of liver damage, and a nail lacquer, which has poor efficacy. The causes for onychomycosis include dermatophytes, non-dermatophyte molds and Candida species. The nail plate may become thickened with yellowish or brownish discoloration, brittle with crumbling edges and, in addition, it is not uncommon for the nail plate to separate from the nail bed. Onychomycosis may be difficult to treat, with high rates of persistence or recurrence of fungal infection.

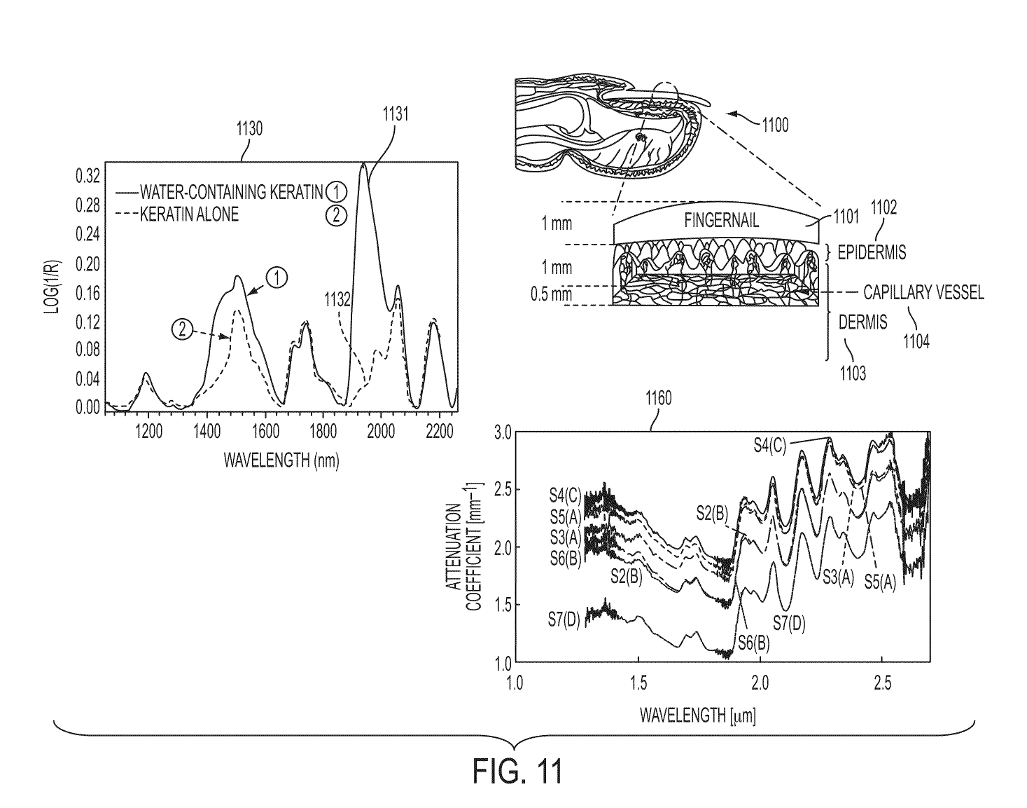

The fungus may grow below the fingernail or toenail, particularly near the beginning section where the nail grows from (e.g., the eponychium or lunula). For laser treatment of the fungus, the light may advantageously penetrate through the fingernail or toenail to the epidermis and dermis below the nail. Thus, it may be desirous to select a wavelength of light that may pass through the nail plate and into the nail bed, which may then result in superheating of the fungal material. It is believed that exposure of the fungi to high temperatures may inhibit their growth as well as cause cell damage, perhaps even cell death. The upper right side of FIG. 11 shows a schematic of the toenail anatomy 1100. The fingernail or toenail 1101 may be about 1 mm in thickness. The next 1 mm may comprise the epidermis 1102 and the top part of the dermis 1103. The top part of the dermis may additionally comprise capillary vessels 1104.

Fingernails and toenails are generally made of tough protein called keratin. In addition, the composition of the nails includes about 7-12% water. Thus, for the wavelength of light to pass the nail plate to the nail bed, it may be desirous to minimize absorption by keratin and water, as well as the scattering through the nail plate. FIG. 11 (left bottom) includes the absorption coefficient 1130 for water-containing keratin 1131 and keratin approximately alone 1132. Water may have strong absorption peaks near about 1450-1500 nm and 1900-2000 nm. In addition, the tough protein keratin has absorption peaks near approximately 1200 nm, 1500 nm, 1700-1750 nm, 2050 nm and 2180 nm. Therefore, it may be advantageous to select laser wavelengths that avoid the keratin and water absorption peaks, such as wavelengths near about 900-1100 nm, 1300 nm, 1650 nm, 1850-1900 nm, or 2100 nm.

Beyond the absorption of keratin, the scattering through the nail plate and overall water absorption may also need to be considered. For example, scattering increases at the shorter wavelengths, since the scattering loss increases inversely as some power of the wavelength. Also, above about 2000 nm, the water absorption background increases with increasing wavelength. As an example, FIG. 11 (right bottom) illustrates the attenuation coefficient (absorption plus scattering) 1160 of nail samples that were allowed to stand in a humidity level of 14%. The infrared spectra are shown for seven samples of nails kept in a saturated aqueous LiCL solution (to control the humidity level at 14%). Several peaks are observed in 1160, which may be peaks of keratin absorption. The baseline for the seven samples may differ due to the influence of scattering. When scattering and absorption are both considered, a global minimum in the attenuation coefficient through the nails may occur near approximately 1850 nm. Therefore, wavelengths near 1850 nm may penetrate optimally into the nail bed for damaging fungi.

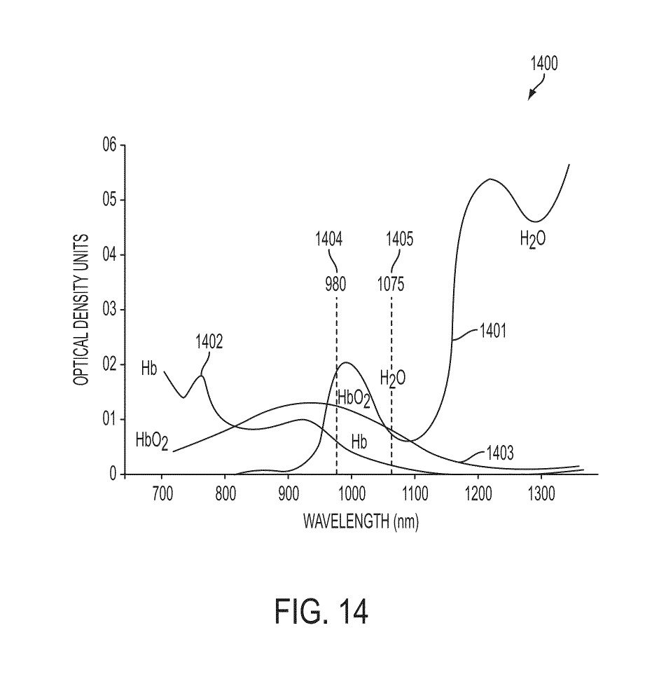

Laser treatment of nail fungus has been studied at near-infrared wavelengths around 1064 nm, 870 nm, and 930 nm. These infrared laser wavelengths may be accompanied by a visible tracer beam, so the physician knows where the light is incident on the sample. However, it may be further advantageous to select a laser wavelength in one of the minima of keratin and water absorption, or near the overall minimum near about 1850 nm. In one embodiment, it may be desirable to select a wavelength near 980 nm 1404 rather than near 1064-1075 nm 1405, as further described with respect to FIG. 14. Also, by focusing the light into the region where the fungal growth occurs rather than in the nail plate, the fungi can be heated with less chance of damage to the nail plate or pain to the patient. Moreover, surface cooling on the skin or nail plate may be used to minimize damage and pain. Although particular embodiments are described, different wavelengths of light may be used, and different combinations of cooling and/or focusing may be used, and these are also intended to be covered by this disclosure.

In yet another embodiment, it may be advantageous to use focused infrared light for treatment of hemorrhoids. A laser hemorrhoidectomy is a procedure that employs laser energy for the treatment of hemorrhoids. When this treatment is applied, a laser may be used to heat the problematic vein, which may in turn cause the vein to collapse and perhaps even disintegrate. Similar to the treatment of varicose veins, focused infrared light may damage the veins while preserving the skin above and in surrounding areas. The focusing of the light as well as possibly using surface cooling procedures may be advantageous for hemorrhoid treatments using lasers. Another advantage of laser hemorrhoidectomy treatment may be that the technique may be used to treat several affected veins at approximately the same time. Furthermore, the laser treatment procedure may minimize the risk of bleeding, which may be a problem in typical surgical procedures. Any combination of the techniques described in this disclosure may be used for the laser treatment of hemorrhoids.

As yet another example, the focused infrared light may be beneficial for laser tissue welding. Bonding of edges of human tissue is a necessary step in most surgical procedures. Current techniques of joining include suture or staple incisions, in which case the tissue bonding process may occur naturally. However, suturing or stapling incisions may introduce foreign materials that may cause inflammation or leave visible scars. On the other hand, laser assisted bonding may help improve post-operative bonding, perhaps even speeding up the healing process.

Laser tissue welding may play a more significant role in surgical methods as laparoscopic, endoscopic and micro-surgical techniques continue to develop. Laser tissue welding may utilize the energy from a laser beam to anastomose tissues, and the technique may be particularly advantageous when suturing or stapling is difficult. In many instances, the laser tissue welding technique also uses a protein solder placed over the anastomosis site. Protein solders may help create a watertight seal, decrease thermal damage, improve consistency of welds, and may even reduce operative times. In one embodiment, laser soldering with albumin based glue may cause less damage and scaring in bonded tissues. For example, experiments have been performed using a 810 nm semiconductor diode laser and two human serum albumin based biomaterials. Experimental evidence also seems to indicate that it may be beneficial to match the optical penetration depth with the thickness of the target tissue. By matching the depth of penetration to tissue thickness, transmural tissue heating may be accomplished.

The mechanisms of laser tissue welding are not precisely known, but there have been many studies that seem consistent with experimental data. Most of these speculations have laser energy being absorbed to create heat-induced changes of the media into collagen, which occurs at temperatures near about 70-80 degrees Celsius. In one example, researchers found that tissue welding resulted in a homogenizing change in the collagen with inter-digitation of the individual fibers. They speculate that the inter-digitation was the structural basis for the tissue welding effect. In another example, researchers found that laser welding led to direct collagen-to-collagen and collagen-to-elastin bonding. In yet another study, researchers report that the mechanism of welding is from the roping effect of parallel collagen fibers as well as the cross-linking that occurs at the cut ends of the collagen fibers. Thus, these studies point to the hypothesis that collagen fiber bonding, through some form of inter-digitation, roping, fusion, or other physiochemical means may be responsible for the welding effect.

Given the importance of collagen in the laser tissue welding process, it may be advantageous to use wavelengths of light that are near an absorption peak in collagen. This may reduce collateral damage near and surrounding the wound healing location. Since the heat goes directly into heating the collagen, this may also reduce the power level required for the laser. The absorption coefficient for collagen 503 is shown in FIG. 5. As an example, there are collagen absorption peaks near 1210 nm 505 and 1720 nm 506, which also correspond to valleys in water absorption, so there would be more direct heating of the collagen and less collateral heating in the tissue with water content. These wavelengths may be particularly advantageous for laser tissue welding. There are other peaks in collagen absorption, such as near 1450 nm and 1950 nm, and these peaks coincide with local peaks in water absorption. There are also other peaks beyond 2000 nm, although the background water absorption increases at these longer wavelengths. Thus, in laser tissue welding if the goal is to minimize collateral damage in surrounding tissue by avoiding water absorption while still heating collagen directly, then selected wavelengths may be near 1210 nm 505 and 1720 nm 506.

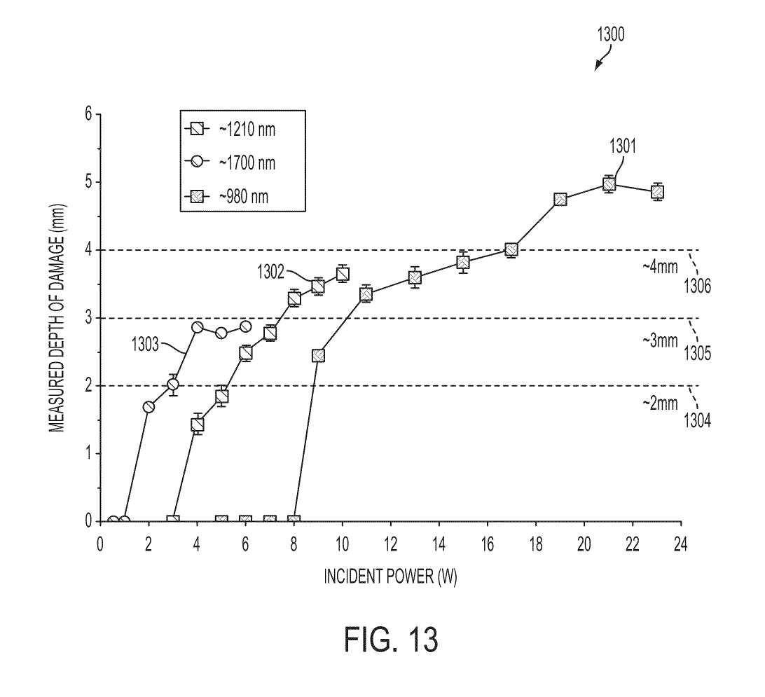

The particular choice of wavelength for laser tissue welding may also be determined by attempting to match the penetration depth to the thickness of the tissue to be welded. For example, FIG. 13 provides experimental measurements of penetration into chicken breast tissue. If the penetration depth is defined as when the damage depth begins to approximately saturate, then for wavelengths near 980 nm 1301 the penetration depth 1306 may be defined as approximately 4 mm. For wavelengths near 1210 nm 1302 the penetration depth 1305 may be defined as approximately 3 mm, and for wavelengths near 1700 nm 1303 the penetration depth 1304 may be defined as approximately 2 mm. These are only approximate values, and other values and criteria may be used to define the penetration depth.

Other modifications may also be used advantageously in the laser tissue welding process, beyond using a wavelength of light near one of the collagen absorption peaks. For example, since the collagen temperature wants to be near or about 70 degrees Celsius, a temperature monitoring system may be used to reduce the laser power when this temperature range is reached. Moreover, it may be advantageous to add a protein solder near or over the anastomosis site, such as albumin solders. When such solders are used, it may also be advantageous to perhaps add a wavelength of light excitation that is absorbed by the solder (e.g., one wavelength might correspond to the collagen, while another wavelength could correspond to the solder. Alternately, there may be a wavelength that is absorbed preferentially by both collagen and the solder). In one embodiment, the solder added could be albumin, and some of the near-infrared absorption peaks for albumin are near approximately 1.51 microns, 1.7 microns, 1.74 microns, 2.17 microns and 2.28 microns. Thus, selecting a wavelength near 1720 nm (1.72 microns) might be absorbed by collagen and albumin. Alternately, beyond the wavelength selected for heating collagen, a second wavelength could be added that falls near one of the albumin absorption peaks. Moreover, using focused light and/or surface cooling techniques may also enable effective laser tissue welding while reducing damage to the top layer, which may result in scaring or unnecessary tissue damage. Although specific embodiments of laser tissue welding are described, other methods and combinations may also be used and are intended to be within the scope of this disclosure.

Although particular examples have been discussed, other therapeutic and diagnostic medical procedures may also benefit for the use of infrared light. Other procedures may benefit from using focused infrared light that may be used to thermally coagulate or occlude relatively shallow vessels non-invasively or minimally invasively while preserving or minimizing damage to the top layer of the skin or tissue. Particularly when these procedures are external to the body, various surface cooling techniques may also be used advantageously. The discussion has been for exemplary applications, but more generally different wavelengths of light may be used, and different combinations of cooling and/or focusing may be used, and these are also covered within the scope of this disclosure.

### Focusing and/or Surface Cooling

Various embodiments of this disclosure provide a method of causing coagulation or occlusion of sections of varicose veins with minimal damage to the skin. One method of achieving this goal may be to focus the light, so that low intensity may be incident on the skin, while higher intensity of light may be incident on the varicose vein wall and lumen. Another method of achieving this goal may be to add surface cooling of the epidermis and dermis, such as using cryogenic spray or liquid-cooled surface contact—techniques that are commonly used in dermatology and cosmetic surgery. In yet another method, some combination of light focusing and surface cooling may be employed. These are provided as particular examples, but other methods of minimizing damage to the skin may also be used and are intended to be covered by this disclosure.

In a non-limiting example, a plurality of spots may be used, or what might be called a fractionated beam. The fractionated laser beam may be added to the laser delivery assembly or delivery head in a number of ways. In one embodiment, a screen-like spatial filter may be placed in the pathway of the beam to be delivered to the biological tissue. The screen-like spatial filter can have opaque regions to block the light and holes or transparent regions, through which the laser beam may pass to the tissue sample. The ratio of opaque to transparent regions may be varied, depending on the application of the laser. In another embodiment, a lenslet array can be used at or near the output interface where the light emerges. In yet another embodiment, at least a part of the delivery fiber from the infrared laser system to the delivery head may be a bundle of fibers, which may comprise a plurality of fiber cores surrounded by cladding regions. The fiber cores can then correspond to the exposed regions, and the cladding areas can approximate the opaque areas not to be exposed to the laser light. As an example, a bundle of fibers may be excited by at least a part of the laser system output, and then the fiber bundle can be fused together and perhaps pulled down to a desired diameter to expose to the tissue sample near the delivery head. In yet another embodiment, a photonic crystal fiber may be used to create the fractionated laser beam. In one non-limiting example, the photonic crystal fiber can be coupled to at least a part of the laser system output at one end, and the other end can be coupled to the delivery head. In a further example, the fractionated laser beam may be generated by a heavily multi-mode fiber, where the speckle pattern at the output may create the high intensity and low intensity spatial pattern at the output. Although several exemplary techniques are provided for creating a fractionated laser beam, other techniques that can be compatible with optical fibers are also intended to be included by this disclosure.

In a further embodiment, it may be advantageous to apply surface cooling techniques to minimize damage to the skin above the varicose veins. In a particular embodiment, the surface cooling may be accomplished by having a thermally conductive surface approximately in contact with the skin, as illustrated 904 in FIG. 9 or 1004 in FIG. 10. Liquid coolant may flow in proximity to the skin, thermally conducting away some of the heat. The cooling fluid may be water, Freon, or other liquids that may have a lower freezing temperature.

In yet another embodiment, the surface cooling may be accomplished using a dynamic cooling device, such as a cryogenic spray. As an example, FIG. 8 illustrates a cooling spray 808 that may be adjacent to the lens/mirror assembly 805 and mounted on a stand 804. In one particular embodiment, the cooling spray 808 may be a dynamic cooling device made by Candela Laser Corporation. As an example, this device may deliver the cryogen (halocarbon 134a, 1,1,1,2-tetrafluoroethane, boiling point=−26 degrees Celsius) to the tissue surface through a solenoid valve. The solenoid valve may be triggered to deliver one or more cryogen pulses to precool the skin before irradiation using the laser. In addition, the cryogen spray may be delivered continuously or intermittently during or between laser pulses, as well as after the laser irradiation is completed. Moreover, a cryogen mask may be employed to thermally insulate the surrounding skin from the cryogen spray, thereby avoiding or minimizing superficial freezing burns. Although a particular embodiment is described, other configurations and combinations of focusing and surface cooling may be used and are intended to be covered by this disclosure.

Beyond the use of focused light and surface cooling, other methods may also be used to reduce the potential for pain or damage to the skin. In yet another embodiment, an optical clearing agent, OCA, may be applied to the skin to reduce the laser power necessary. The OCA may reduce skin scattering and increase transmission through the skin, thereby reducing the required power levels and the risk of skin burns. The OCA may also reduce the differences in refractive index between different skin layers and air, thereby reducing the amount of reflected light from refractive index mismatches. Examples of common OCAs include dimethyl sulfoxide, glycerol, glucose and other sugar compounds—as well as mixtures of these compounds. Also, in one embodiment the OCA may be delivered to the skin using a pneumatic jet device, such as a Madajet device made by Advanced Meditech International. For instance, the OCA may be applied near and around the spot(s) of laser irradiation.

In another embodiment, a local anesthetic may be used in the vicinity of the laser irradiation and clamp or mount. One example of a local anesthesia may be lidocaine. Many local anesthetics may be membrane stabilizing drugs, and local anesthetics may be bases and may usually be formulated as the hydrochloride salt to render them water-soluble. Beyond optical clearing agents and local anesthesia, other ointments, creams, liquids or sprays may also be applied to the skin area before, during and after the laser irradiation, and these are also intended to be covered by this disclosure.

### Laser Experiments

### Penetration Depth, Focusing, Skin Cooling

Some preliminary experiments show the feasibility of using focused infrared light for non-invasive varicose vein procedures, or other procedures where relatively shallow vessels below the skin are to be thermally coagulated or occluded with minimum damage to the skin upper layers. In one embodiment, the penetration depth and optically induced thermal damage has been studied in chicken breast samples. Chicken breast may be a reasonable optical model for smooth muscle tissue, comprising water, collagen and proteins. Commercially available chicken breast samples were kept in a warm bath (about 32 degrees Celsius) for about an hour, and then about half an hour at room temperature in preparation for the measurements.

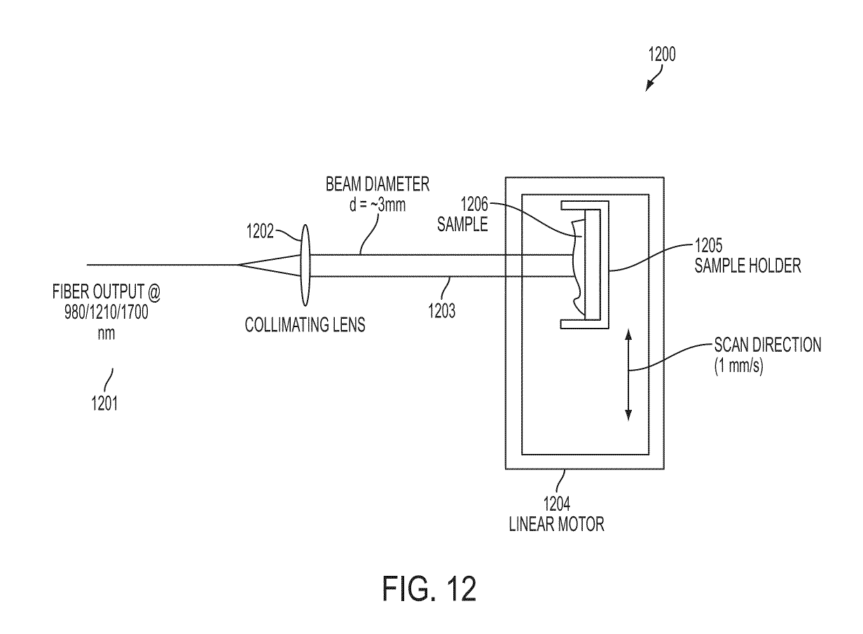

An exemplary set-up 1200 for testing chicken breast samples using collimated light is illustrated in FIG. 12. The laser light 1201 near 980 nm, 1210 nm, or 1700 nm may be provided from one or more laser diodes or fiber lasers, as described further herein. In this instance, laser diodes were used, which comprise a plurality of laser diode emitters that are combined using one or more multiplexers (particularly spatial multiplexers), and then the combined beam is coupled into a multi-mode fiber (typically 100 microns to 400 microns in diameter). The output from the laser diode fiber was then collimated using one or more lenses 1202. The resulting beam 1203 was approximately round with a beam diameter of about 3 mm. The beam diameter was verified by blade measurements (i.e., translating a blade across the beam). Also, the time-averaged power was measured in the nearly collimated section after the lens using a large power meter. The chicken breast samples 1206 were mounted in a sample holder 1205, and the sampler holder 1205 was mounted in turn on a translation stage 1204 with a linear motor that could move perpendicular to the incoming laser beam. Although particular details of the experiment are described, other elements may be added or eliminated, and these alternate embodiments are also intended to be covered by this disclosure.

For these particular experiments, the measured depth of damage (in millimeters) versus the incident laser power (in Watts) is shown 1300 in FIG. 13. In this embodiment, laser diodes were used at wavelengths near 980 nm, 1210 nm, and 1700 nm. The curve 1301 corresponds to wavelengths near 980 nm, the curve 1302 corresponds to wavelengths near 1210 nm, and the curve 1303 corresponds to wavelengths near 1700 nm. It may be noted that there is a threshold power, above which the damage depth increases relatively rapidly. For example, the threshold power for wavelengths near 980 nm may be about 8 W, the threshold power for wavelengths near 1210 nm may be 3 W, and the threshold power for wavelengths near 1700 nm may be about 1 W. The threshold powers may be different at the different wavelengths because of the difference in water absorption (e.g., 501 in FIG. 5). Part of the difference in threshold powers may also arise from the absorption of proteins such as collagen (e.g., 503 in FIG. 5). After a certain power level, the damage depth appears to saturate: i.e., the slope flattens out as a function of increasing pump power.

In one embodiment, if the penetration depth is defined as when the damage begins to approximately saturate, then for wavelengths near 980 nm 1301 the penetration depth 1306 may be defined as approximately 4 mm. For wavelengths near 1210 nm 1302 the penetration depth 1305 may be defined as approximately 3 mm, and for wavelengths near 1700 nm 1303 the penetration depth 1304 may be defined as approximately 2 mm. These are only approximate values, and other values and criteria may be used to define the penetration depth. It may also be noted that the level of damage at the highest power points differs at the different wavelengths. For example, at the highest power point of 1303 near 1700 nm, much more damage is observed, showing evidence of even boiling and cavitation. This may be due to the higher absorption level near 1700 nm (e.g., 501 in FIG. 5). On the other hand, at the highest power point 1301 near 980 nm, the damage is not as catastrophic, but the spot size appears larger. The larger spot size may be due to the increased scattering at the shorter wavelengths (e.g., 501 in FIG. 5). Data 1300 such as in FIG. 13 may be used to select the particular wavelength for the laser beam to be used in the non-invasive procedure.

Even near wavelengths such as described in FIG. 13, the particular wavelength selected may be more specifically defined based on the target tissue of interest. In one particular embodiment, the vessel lumen may be modeled as water, and for this example assume that wavelengths in the vicinity of 980 nm are being selected to create thermal coagulation or occlusion.

FIG. 14 shows the optical absorption or density as a function of wavelength 1400 between approximately 700 nm and 1300 nm. Curves are shown for the water absorption 1401, hemoglobin Hb absorption 1402, and oxygenated hemoglobin HbO₂ 1403. In this example, two particular wavelengths are compared: 980 nm 1404 and 1075 nm 1405. For instance, 980 nm may be generated using one or more laser diodes, while 1075 nm may be generated using an ytterbium-doped fiber laser. If maximizing the penetration depth is desired, then 1075 nm 1405 may be selected, since it falls near a local minimum in water 1401, hemoglobin 1402 and oxygenated hemoglobin 1403 absorption. On the other hand, if the penetration depth at 980 nm 1404 is adequate and the goal is to generate heat through water absorption, then 980 nm 1404 may be a selected wavelength for the light source because of the higher water absorption. This wavelength range is only meant to be exemplary, but other wavelength ranges and particular criteria for selecting the wavelength may be used and are intended to be covered by this disclosure.

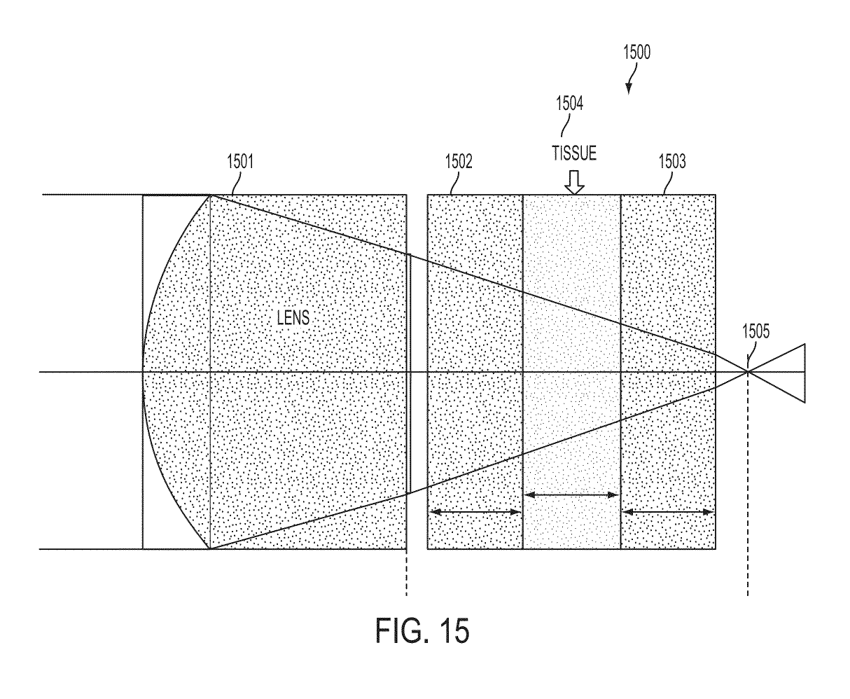

In another embodiment, focused infrared light has been used to preserve the top layer of a tissue while damaging nerves at a deeper level. For instance, FIG. 15 illustrates the set-up 1500 used for the focused infrared experiments. In this embodiment, a lens 1501 is used to focus the light. Although a single lens is shown, either multiple lenses, GRIN (graduated index) lenses, curved mirrors, or a combination of lenses and mirrors may be used. In this particular example, the tissue 1504 is placed between two microscope slides 1502 and 1503 for in vitro experiments. The tissue 1504 is renal artery wall tissue either from porcine or bovine animals (about 1.2 mm thick sample)—i.e., this is the artery leading to the kidneys, and it is the artery where typically renal denervation may be performed to treat hypertension. For this example, the minimum beam waist 1505 falls behind the tissue, and the intensity contrast from the front of the tissue (closest to the lens) to the back of the tissue (furthest from the lens) is about 4:1. These are particular ranges used for this experiment, but other values and locations of minimum beam waist may also be used and are intended to be covered by this disclosure.

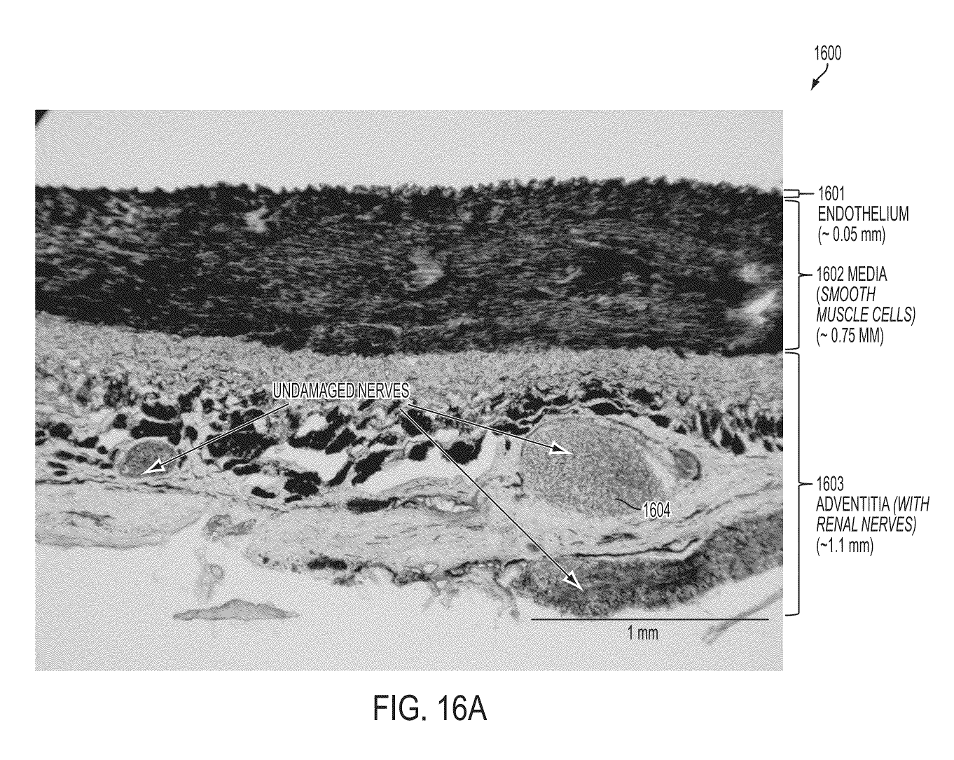

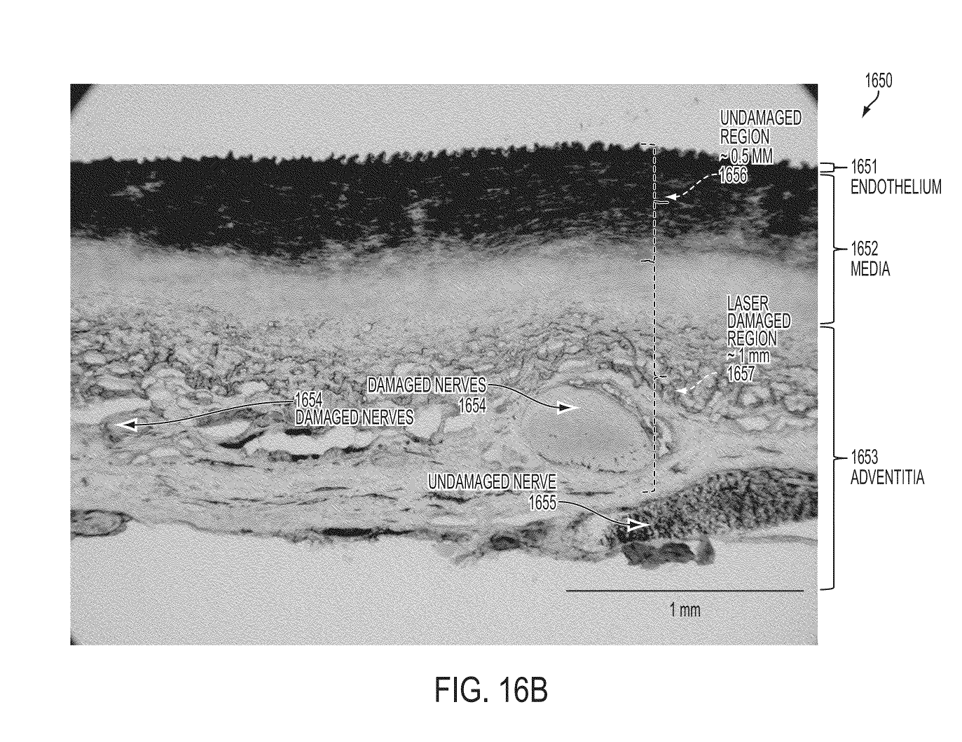

For a particular embodiment, histology of the renal artery is shown in FIG. 16A for no laser exposure 1600 and shown in FIG. 16B with focused infrared laser exposure 1650. In this experiment, the beam diameter incident on the lens was about 4 mm, and the distance from the edge of the flat side of lens to the minimum beam waist was about 3.75 mm. The beam diameter on the front side of the renal artery (i.e., the endothelium side) was about 1.6 mm, and the beam diameter on the back side of the renal artery was about 0.8 mm. In FIG. 16A with no laser exposure, the layers of the artery wall may be identified: top layer of endothelium 1601 that is about 0.05 mm thick, the media comprising smooth muscle cells or tissue 1602 that is about 0.75 mm thick, and the adventitia 1603 comprising some of the renal nerves 1604 that is about 1.1 mm thick. These are particular values for this experiment, and other layers and thicknesses may also be used and are intended to be covered by this disclosure.

The histology with focused infrared light exposure 1650 is illustrated in FIG. 16B. The laser light used is near 1708 nm from a cascaded Raman oscillator (described in greater detail herein), and the power incident on the tissue is about 0.8 W and the beam is scanned across the tissue at an approximately 0.4 mm/sec rate. The various layers are still observable: the endothelium 1651, the media 1652, and the adventitia 1653. With this type of histology, the non-damaged regions remain darker (similar to FIG. 16A), while the laser induced damaged regions turn lighter in color. In this example, the endothelium 1651 and top layer of the media 1652 remain undamaged—i.e., the top approximately 0.5 mm is the undamaged region 1656. The laser damaged region 1657 extends for about 1 mm, and it includes the bottom layer of the media 1652 and much of the adventitia 1653. The renal nerves 1654 that fall within the damage region 1657 are also damaged (i.e., lighter colored). On the other hand, the renal nerves beyond this depth, such as 1655, may remain undamaged.

Thus, by using focused infrared light near 1708 nm in this example, the top approximately 0.5 mm of the renal artery is spared from laser damage. It should be noted that when the same experiment is conducted with a collimated laser beam, then the entire approximately 1.5 mm is damaged (i.e, including regions 1656 and 1657). Therefore, the cone of light with the lower intensity at the top and the higher intensity toward the bottom may, in fact, help preserve the top layer from damage. There should be a Beer's Law attenuation of the light intensity as the light propagates into the tissue. For example, the light intensity should reduce exponentially at a rate determined by the absorption coefficient. In these experiments it appears that the focused light is able to overcome the Beer's law attenuation and still provide contrast in intensity between the front and back surfaces.

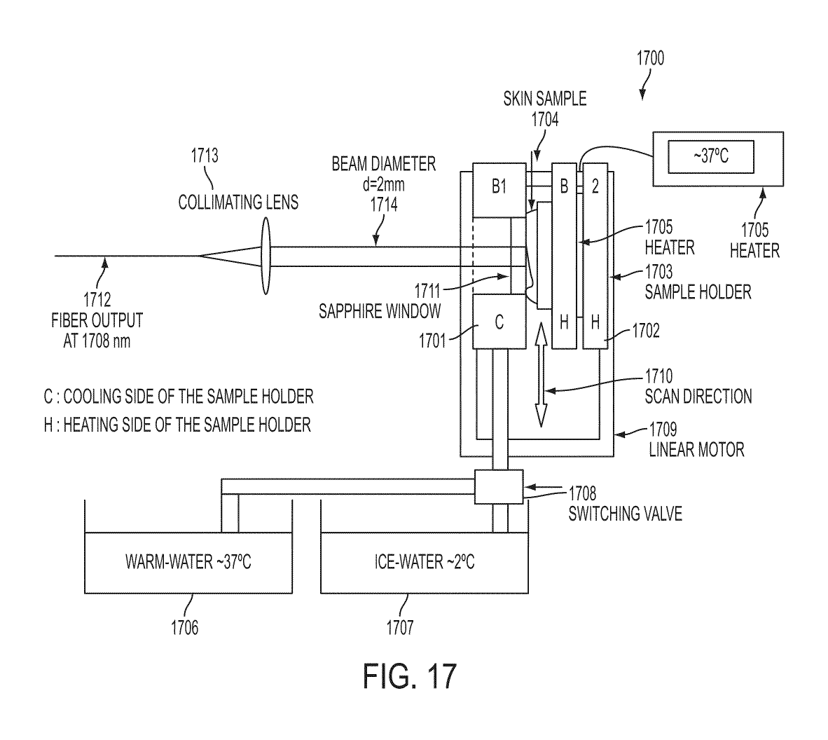

In another embodiment, experiments have also been conducted on dermatology samples with surface cooling, and surface cooling was shown to preserve the top layer of the skin during laser exposure. In this particular example, the experimental set-up 1700 is illustrated in FIG. 17. The skin sample 1704, or more generally sample under test, is placed in a sample holder 1703. The sample holder 1703 has a cooling side 1701 and a heating side 1702. The heating side 1702 comprises a heater 1705, which may be adjusted to operate around 37 degrees Celsius—i.e., close to body temperature. The cooling side 1701 is coupled to an ice-water bath 1707 (around 2 degrees Celsius) and a warm-water bath 1706 (around 37 degrees Celsius) through a switching valve 1708. The entire sample holder 1703 is mounted on a linear motor 1709, so the sample can be moved perpendicular 1710 to the incoming light beam.

In this embodiment, the light is incident on the sample 1704 through a sapphire window 1711. The sapphire material 1711 is selected because it is transparent to the infrared wavelengths, while also being a good thermal conductor. Thus, the top layer of the sample 1704 may be cooled by being approximately in contact with the sapphire window 1711. The laser light 1712 used is near 1708 nm from a cascaded Raman oscillator (described in greater detail herein), and one or more collimating lenses 1713 are used to create a beam with a diameter 1714 of approximately 2 mm. This is one particular embodiment of the sample surface cooling arrangement, but other apparatuses and methods may be used and are intended to be covered by this disclosure.

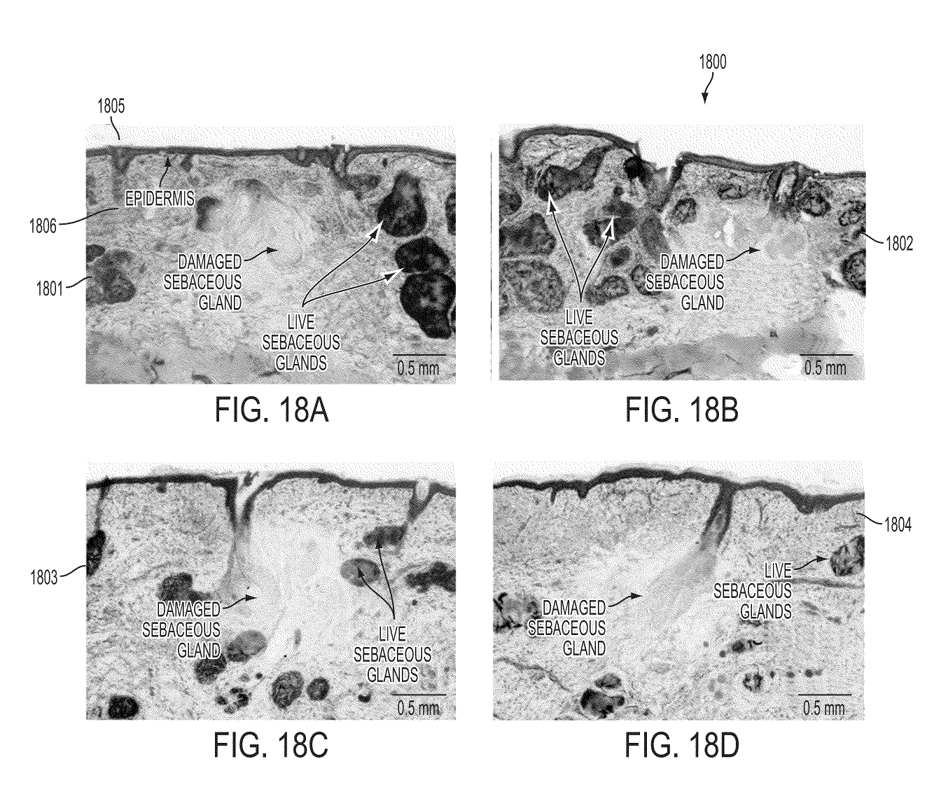

Experimental results obtained using the set-up of FIG. 17 are included in FIG. 18. In this example, FIG. 18 shows the MTT histochemistry of human skin 1800 treated with wavelengths near 1708 nm generated by a laser (5 seconds pre-cool; 2 mm diameter spot exposure for 3 seconds) at 725 mW (A 1801, B 1802) corresponding to about 70 J/cm² average fluence, and 830 mW (C 1803, D 1804) corresponding to about 80 J/cm² average fluence. The images in FIG. 18 show that the application of a cold window was effective in protecting the epidermis 1805 (darker top layer) and the top approximately 0.4-0.5 mm of the dermis 1806. As before, the darker regions of the histology correspond to undamaged regions, while the lighter regions correspond to damaged regions. In contrast, when no surface cooling is applied, then thermal damage to the dermis occurs in the epidermis and dermis where the laser exposure occurs, and the thermal damage extends to about 1.3-1.4 mm or more from the skin surface. Thus, surface cooling applied to the skin may help to reduce or eliminate damage to the top layer of the skin under laser exposure.

In summary, experiments verify that infrared light, such as near 980 nm, 1210 nm, or 1700 nm, may achieve penetration depths between approximately 2 mm to 4 mm or more. The top layer of skin or tissue may be spared damage under laser exposure by focusing the light, applying surface cooling, or some combination of the two. These are particular experimental results, but other wavelengths, methods and apparatuses may be used for achieving the penetration and minimizing damage to the top layer and are intended to be covered by this disclosure. In an alternate embodiment, it may be beneficial to use wavelengths near 1310 nm if the absorption from skin constituents (FIG. 5), such as collagen 503, adipose 502 and elastin 504, are to be minimized. The water absorption 501 near 1310 nm may still permit a penetration depth of approximately 1 cm, or perhaps less. In yet another embodiment, wavelengths near 1210 nm may be beneficial, if penetration depths on the order of 3 mm are adequate and less scattering loss (e.g. 501 in FIG. 5) is desired. Any of FIG. 5 or 13 may be used to select these or other wavelengths to achieve the desired penetration depth and to also perhaps target particular tissue of interest, and these alternate embodiments are also intended to be covered by this disclosure.

### Laser Systems for Therapeutics or Diagnostics

Infrared light sources can be used for diagnostics and therapeutics in a number of medical applications. For example, broadband light sources can advantageously be used for diagnostics, while narrower band light sources can advantageously be used for therapeutics. In one embodiment, selective absorption or damage can be achieved by choosing the laser wavelength to lie approximately at an absorption peak of particular tissue types. Also, by using infrared wavelengths that minimize water absorption peaks and longer wavelengths that have lower tissue scattering, larger penetration depths into the biological tissue can be obtained. In this disclosure, infrared wavelengths are defined as wavelengths in the range of approximately 0.9 microns to 10 microns, more preferably wavelengths between about 0.98 and 2.5 microns.

As used throughout this disclosure, the term “couple” and or “coupled” refers to any direct or indirect communication between two or more elements, whether or not those elements are physically connected to one another. In this disclosure, the term “damage” refers to affecting a tissue or sample so as to render the tissue or sample inoperable. For instance, if a particular tissue normally emits certain signaling chemicals, then by “damaging” the tissue is meant that the tissue reduces or no longer emits that certain signaling chemical. The term “damage” and or “damaged” may include ablation, melting, charring, killing, or simply incapacitating the chemical emissions from the particular tissue or sample. In one embodiment, histology or histochemical analysis may be used to inspect and determine whether a tissue or sample has been damaged.

As used throughout this disclosure, the term “spectroscopy” means that a tissue or sample is inspected by comparing different features, such as wavelength (or frequency), spatial location, transmission, absorption, reflectivity, scattering, refractive index, or opacity. In one embodiment, “spectroscopy” may mean that the wavelength of the light source is varied, and the transmission, absorption or reflectivity of the tissue or sample is measured as a function of wavelength. In another embodiment, “spectroscopy” may mean that the wavelength dependence of the transmission, absorption or reflectivity is compared between different spatial locations on a tissue or sample. As an illustration, the “spectroscopy” may be performed by varying the wavelength of the light source, or by using a broadband light source and analyzing the signal using a spectrometer, wavemeter, or optical spectrum analyzer.

As used throughout this document, the term “fiber laser” refers to a laser or oscillator that has as an output light or an optical beam, wherein at least a part of the laser comprises an optical fiber. For instance, the fiber in the “fiber laser” may comprise one of or a combination of a single mode fiber, a multi-mode fiber, a mid-infrared fiber, a photonic crystal fiber, a doped fiber, a gain fiber, or, more generally, an approximately cylindrically shaped waveguide or light-pipe. In one embodiment, the gain fiber may be doped with rare earth material, such as ytterbium, erbium, and/or thulium. In another embodiment, the infrared fiber may comprise one or a combination of fluoride fiber, ZBLAN fiber, chalcogenide fiber, tellurite fiber, or germanium doped fiber. In yet another embodiment, the single mode fiber may include standard single-mode fiber, dispersion shifted fiber, non-zero dispersion shifted fiber, high-nonlinearity fiber, and small core size fibers.

As used throughout this disclosure, the term “pump laser” refers to a laser or oscillator that has as an output light or an optical beam, wherein the output light or optical beam may be coupled to a gain medium to excite the gain medium, which in turn may amplify another input optical signal or beam. In one particular example, the gain medium may be a doped fiber, such as a fiber doped with ytterbium, erbium, and/or thulium. In another embodiment, the gain medium may be a fused silica fiber or a fiber with a Raman effect from the glass. In one embodiment, the “pump laser” may be a fiber laser, a solid state laser, a laser involving a nonlinear crystal, an optical parametric oscillator, a semiconductor laser, or a plurality of semiconductor lasers that may be multiplexed together. In another embodiment, the “pump laser” may be coupled to the gain medium by using a fiber coupler, a dichroic mirror, a multiplexer, a wavelength division multiplexer, a grating, or a fused fiber coupler.

As used throughout this document, the term “super-continuum” and/or “supercontinuum” and/or “SC” refers to a broadband light beam or output that comprises a plurality of wavelengths. In a particular example, the plurality of wavelengths may be adjacent to one-another, so that the spectrum of the light beam or output appears as a continuous band when measured with a spectrometer. In one embodiment, the broadband light beam may have a bandwidth of at least 10 nm. In another embodiment, the “super-continuum” may be generated through nonlinear optical interactions in a medium, such as an optical fiber or nonlinear crystal. For example, the “super-continuum” may be generated through one or a combination of nonlinear activities such as four-wave mixing, the Raman effect, modulational instability, and self-phase modulation.

As used throughout this disclosure, the terms “optical light” and/or “optical beam” and or “light beam” refer to photons or light transmitted to a particular location in space. The “optical light” and or “optical beam” and/or “light beam” may be modulated or unmodulated, which also means that they may or may not contain information. In one embodiment, the “optical light” and/or “optical beam” and/or “light beam” may originate from a fiber, a fiber laser, a laser, a light emitting diode, a lamp, a pump laser, or a light source.

As used throughout this document, the terms “near”, “about”, and the symbol “˜” are used to designate approximate center wavelengths with a range that may depend on the particular application. For example, in one embodiment “about 1720 nm” refers to one or more wavelengths of light with a wavelength value anywhere between approximately 1680 nm and 1760 nm. In another embodiment, the term “near 1720 nm” refers to one or more wavelengths of light with a wavelength value anywhere between approximately 1700 nm and 1740 nm. Similarly, as used throughout this document, the term “near 1210 nm” may refer to one or more wavelengths of light with a wavelength value anywhere between approximately 1170 nm and 1250 nm. In one embodiment, the term “near 1210 nm” refers to one or more wavelengths of light with a wavelength value anywhere between approximately 1190 nm and 1230 nm.

Different light sources may be selected for the infrared based on the needs of the application. Some of the features for selecting a particular light source include power or intensity, wavelength range or bandwidth, spatial or temporal coherence, spatial beam quality for focusing or transmission over long distance, and pulse width or pulse repetition rate. Depending on the application, lamps, light emitting diodes (LEDs), laser diodes (LD's), tunable LD's, super-luminescent laser diodes (SLDs), fiber lasers or super-continuum sources (SC) may be advantageously used. Also, different fibers may be used for transporting the light, such as fused silica fibers, plastic fibers, mid-infrared fibers (e.g., tellurite, chalcogenides, fluorides, ZBLAN, etc), photonic crystal fibers, or a hybrid of these fibers.

In one embodiment, LED's can be used that have a higher power level in the infrared wavelength range. LED's produce an incoherent beam, but the power level can be higher than a lamp and with higher energy efficiency. Also, the LED output may more easily be modulated, and the LED provides the option of continuous wave or pulsed mode of operation. LED's are solid state components that emit a wavelength band that is of moderate width, typically between about 20 nm to 40 nm. There are also so-called super-luminescent LEDs that may even emit over a much wider wavelength range. In another embodiment, a wide band light source may be constructed by combining different LEDs that emit in different wavelength bands, some of which could overlap in spectrum. One advantage of LEDs as well as other solid state components is the compact size that they may be packaged into.

In yet another embodiment, various types of laser diodes may be used in the infrared wavelength range. Just as LEDs may be higher in power but narrower in wavelength emission than lamps and thermal sources, the LDs may be yet higher in power but yet narrower in wavelength emission than LEDs. Different kinds of LDs may be used, including Fabry-Perot LDs, distributed feedback (DFB) LDs, distributed Bragg reflector (DBR) LDs. A plurality of LDs may be spatially multiplexed, polarization multiplexed, wavelength multiplexed, or a combination of these multiplexing methods. Also, the LDs may be fiber pig-tailed or have one or more lenses on the output to collimate or focus the light. Another advantage of LDs is that they may be packaged compactly and may have a spatially coherent beam output. Moreover, tunable LDs that can tune over a range of wavelengths are also available. The tuning may be done by varying the temperature, or electrical current may be used in particular structures such as distributed Bragg reflector LDs. In another embodiment, external cavity LDs may be used that have a tuning element, such as a fiber grating or a bulk grating, in the external cavity.

In another embodiment, super-luminescent laser diodes may provide higher power as well as broad bandwidth. An SLD is typically an edge emitting semiconductor light source based on super-luminescence (e.g., this could be amplified spontaneous emission). SLDs combine the higher power and brightness of LDs with the low coherence of conventional LEDs, and the emission band for SLD's may be 5 nm to 100 nm wide, preferably in the 60 nm to 100 nm range. Although currently SLDs are commercially available in the wavelength range of approximately 400 nm to 1700 nm, SLDs could and may in the future be made the cover a broader region of the infrared.

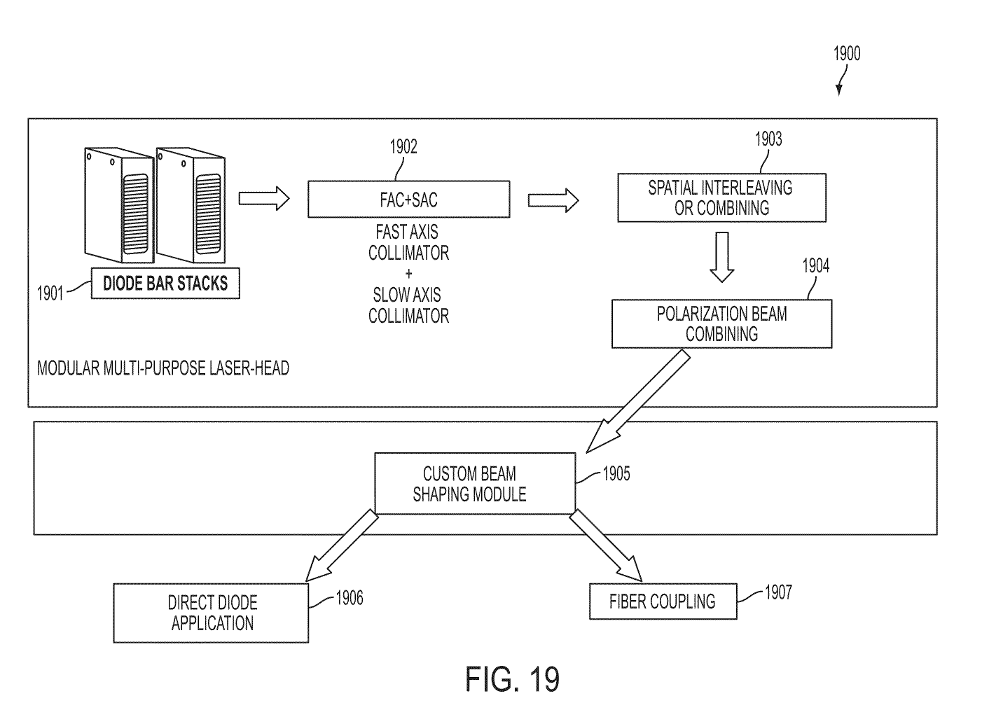

In yet another embodiment, high power LDs for either direct excitation or to pump fiber lasers and SC light sources may be constructed using one or more laser diode bar stacks. As an example, FIG. 19 shows an example of the block diagram 1900 or building blocks for constructing the high power LDs. In this embodiment, one or more diode bar stacks 1901 may be used, where the diode bar stack may be an array of several single emitter LDs. Since the fast axis (e.g., vertical direction) may be nearly diffraction limited while the slow-axis (e.g., horizontal axis) may be far from diffraction limited, different collimators 1902 may be used for the two axes.

Brightness may be increased by spatially combining the beams from multiple stacks 1903. The combiner may include spatial interleaving, wavelength multiplexing, or a combination of the two. Different spatial interleaving schemes may be used, such as using an array of prisms or mirrors with spacers to bend one array of beams into the beam path of the other. In another embodiment, segmented mirrors with alternate high-reflection and anti-reflection coatings may be used. Moreover, the brightness may be increased by polarization beam combining 1904 the two orthogonal polarizations, such as by using a polarization beam splitter. In a particular embodiment, the output may then be focused or coupled into a large diameter core fiber. As an example, typical dimensions for the large diameter core fiber range from diameters of approximately 100 microns to 400 microns or more. Alternatively or in addition, a custom beam shaping module 1905 may be used, depending on the particular application. For example, the output of the high power LD may be used directly 1906, or it may be fiber coupled 1907 to combine, integrate, or transport the high power LD energy. These high power LDs may grow in importance because the LD powers can rapidly scale up. For example, instead of the power being limited by the power available from a single emitter, the power may increase in multiples depending on the number of diodes multiplexed and the size of the large diameter fiber. Although FIG. 19 is shown as one embodiment, some or all of the elements may be used in a high power LD, or additional elements may also be used.

### Infrared Super-Continuum Lasers

Each of the light sources described above have particular strengths, but they also may have limitations. For example, there is typically a trade-off between wavelength range and power output. Also, sources such as lamps, thermal sources, and LEDs produce incoherent beams that may be difficult to focus to a small area and may have difficulty propagating for long distances. An alternative source that may overcome some of these limitations is an SC light source. Some of the advantages of the SC source may include high power and intensity, wide bandwidth, spatially coherent beam that can propagate nearly transform limited over long distances, and easy compatibility with fiber delivery.

Supercontinuum lasers may combine the broadband attributes of lamps with the spatial coherence and high brightness of lasers. By exploiting a modulational instability initiated supercontinuum (SC) mechanism, an all-fiber-integrated SC laser with no moving parts may be built using commercial-off-the-shelf (COTS) components. Moreover, the fiber laser architecture may be a platform where SC in the visible, near-infrared/SWIR, or mid-IR can be generated by appropriate selection of the amplifier technology and the SC generation fiber. But until now, SC lasers were used primarily in laboratory settings since typically large, table-top, mode-locked lasers were used to pump nonlinear media such as optical fibers to generate SC light. However, those large pump lasers may now be replaced with diode lasers and fiber amplifiers that gained maturity in the telecommunications industry.

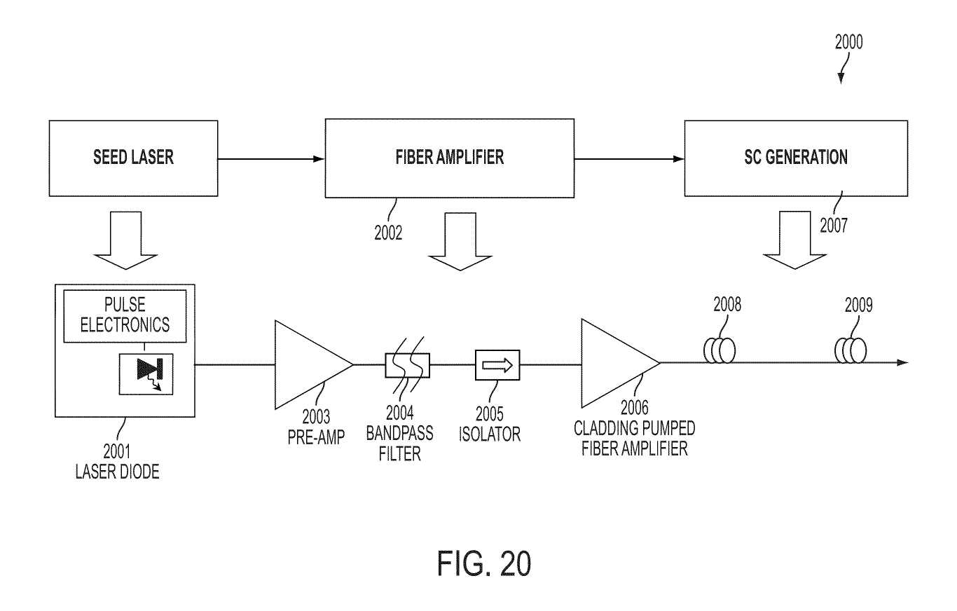

In one embodiment, an all-fiber-integrated, high-powered SC light source 2000 may be elegant for its simplicity (FIG. 20). The light may be first generated from a seed laser diode 2001. For example, the seed LD 2001 may be a distributed feedback (DFB) laser diode with a wavelength near 1542 nm or 1550 nm, with approximately 0.5-2.0 ns pulsed output, and with a pulse repetition rate between one kilohertz and about 100 MHz or more. The output from the seed laser diode may then be amplified in a multiple-stage fiber amplifier 2002 comprising one or more gain fiber segments. In a particular embodiment, the first stage pre-amplifier 2003 may be designed for optimal noise performance. For example, the pre-amplifier 2003 may be a standard erbium-doped fiber amplifier or an erbium/ytterbium doped cladding pumped fiber amplifier. Between amplifier stages 2003 and 2006, it may be advantageous to use band-pass filters 2004 to block amplified spontaneous emission and isolators 2005 to prevent spurious reflections. Then, the power amplifier stage 2006 may use a cladding-pumped fiber amplifier that may be optimized to minimize nonlinear distortion. The power amplifier fiber 2006 may also be an erbium-doped fiber amplifier, if only low or moderate power levels are to be generated.

The SC generation 2007 may occur in the relatively short lengths of fiber that follow the pump laser. Exemplary SC fiber lengths may range from a few millimeters to 100 m or more. In one embodiment, the SC generation may occur in a first fiber 2008 where the modulational-instability initiated pulse break-up occurs primarily, followed by a second fiber 2009 where the SC generation and spectral broadening occurs primarily.

In one embodiment, one or two meters of standard single-mode fiber (SMF) after the power amplifier stage may be followed by several meters of SC generation fiber. For this example, in the SMF the peak power may be several kilowatts and the pump light may fall in the anomalous group-velocity dispersion regime—often called the soliton regime. For high peak powers in the dispersion regime, the nanosecond pulses may be unstable due to a phenomenon known as modulational instability, which is basically parametric amplification in which the fiber nonlinearity helps to phase match the pulses. As a consequence, the nanosecond pump pulses may be broken into many shorter pulses as the modulational instability tries to form soliton pulses from the quasi-continuous-wave background. Although the laser diode and amplification process starts with approximately nanosecond-long pulses, modulational instability in the short length of SMF fiber may form approximately 0.5 ps to several-picosecond-long pulses with high intensity. Thus, the few meters of SMF fiber may result in an output similar to that produced by mode-locked lasers, except in a much simpler and cost-effective manner.

The short pulses created through modulational instability may then be coupled into a nonlinear fiber for SC generation. The nonlinear mechanisms leading to broadband SC may include four-wave mixing or self-phase modulation along with the optical Raman effect. Since the Raman effect is self-phase-matched and shifts light to longer wavelengths by emission of optical photons, the SC may spread to longer wavelengths very efficiently. The short-wavelength edge may arise from four-wave mixing, and often times the short wavelength edge may be limited by increasing group-velocity dispersion in the fiber. In many instances, if the particular fiber used has sufficient peak power and SC fiber length, the SC generation process may fill the long-wavelength edge up to the transmission window.

Mature fiber amplifiers for the power amplifier stage 2006 include ytterbium-doped fibers (near 1060 nm), erbium-doped fibers (near 1550 nm), erbium/ytterbium-doped fibers (near 1550 nm), or thulium-doped fibers (near 2000 nm). In various embodiments, candidates for SC fiber 2009 include fused silica fibers (for generating SC between 0.8-2.7 μm), mid-IR fibers such as fluorides, chalcogenides, or tellurites (for generating SC out to 4.5 μm or longer), photonic crystal fibers (for generating SC between 0.4 and 1.7 μm), or combinations of these fibers. Therefore, by selecting the appropriate fiber-amplifier doping for 2006 and nonlinear fiber 2009, SC may be generated in the visible, near-IR/SWIR, or mid-IR wavelength region.

The configuration 2000 of FIG. 20 is just one particular example, and other configurations can be used and are intended to be covered by this disclosure. For example, further gain stages may be used, and different types of lossy elements or fiber taps may be used between the amplifier stages. In another embodiment, the SC generation may occur partially in the amplifier fiber and in the pig-tails from the pump combiner or other elements. In yet another embodiment, polarization maintaining fibers may be used, and a polarizer may also be used to enhance the polarization contrast between amplifier stages. Also, not discussed in detail are many accessories that may accompany this set-up, such as driver electronics, pump laser diodes, safety shut-offs, and thermal management and packaging.

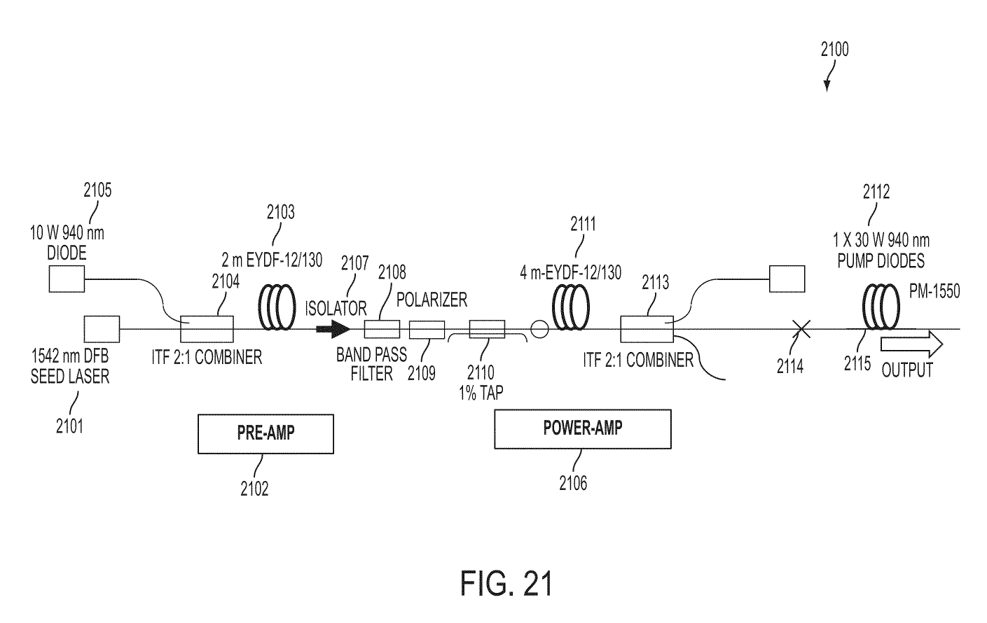

In one embodiment, one example of the SC laser that operates in the short wave infrared (SWIR) is illustrated in FIG. 21. This SWIR SC source 2100 produces an output of up to approximately 5 W over a spectral range of about 1.5 microns to 2.4 microns, and this particular laser is made out of polarization maintaining components. The seed laser 2101 is a distributed feedback (DFB) laser operating near 1542 nm producing approximately 0.5 nsec pulses at an about 8 MHz repetition rate. The pre-amplifier 2102 is forward pumped and uses about 2 m length of erbium/ytterbium cladding pumped fiber 2103 (often also called dual-core fiber) with an inner core diameter of 12 microns and outer core diameter of 130 microns. The pre-amplifier gain fiber 2103 is pumped using a 10 W 940 nm laser diode 2105 that is coupled in using a fiber combiner 2104.

In this particular 5 W unit, the mid-stage between amplifier stages 2102 and 2106 comprises an isolator 2107, a band-pass filter 2108, a polarizer 2109 and a fiber tap 2110. The power amplifier 2106 uses a 4 m length of the 12/130 micron erbium/ytterbium doped fiber 2111 that is counter-propagating pumped using one or more 30 W 940 nm laser diodes 2112 coupled in through a combiner 2113. An approximately 1-2 meter length of the combiner pig-tail helps to initiate the SC process, and then a length of PM-1550 fiber 2115 (polarization maintaining, single-mode, fused silica fiber optimized for 1550 nm) is spliced 2114 to the combiner output.

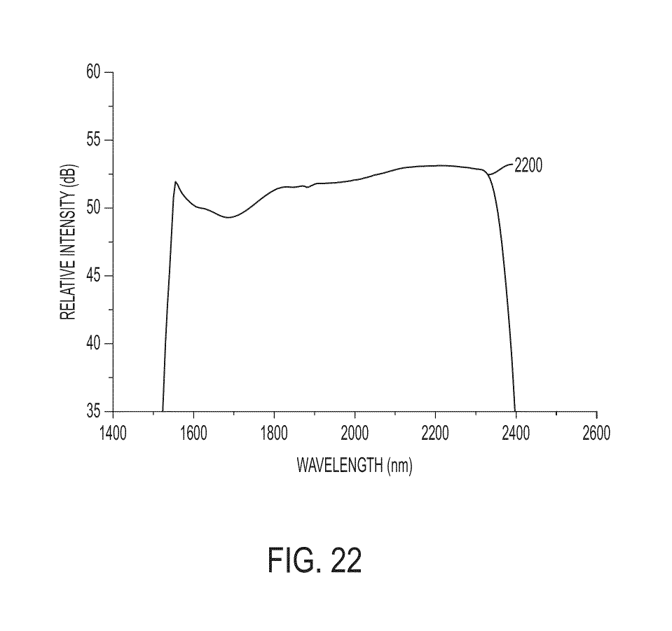

If an approximately 10 m length of output fiber is used, then the resulting output spectrum 2200 is shown in FIG. 22. The details of the output spectrum 2200 depend on the peak power into the fiber, the fiber length, and properties of the fiber such as length and core size, as well as the zero dispersion wavelength and the dispersion properties. For example, if a shorter length of fiber is used, then the spectrum actually reaches to longer wavelengths (e.g., a 2 m length of SC fiber broadens the spectrum to about 2500 nm). Also, if extra-dry fibers are used with less O—H content, then the wavelength edge may also reach to a longer wavelength. To generate more spectrum toward the shorter wavelengths, the pump wavelength (in this case ˜1542 nm) should be close to the zero dispersion wavelength in the fiber. For example, by using a dispersion shifted fiber or so-called non-zero dispersion shifted fiber, the short wavelength edge may shift to shorter wavelengths.

Although one particular example of a 5 W SWIR-SC implementation has been described, different components, different fibers, and different configurations may also be used consistent with this disclosure. For instance, another embodiment of the similar configuration 2100 in FIG. 21 may be used to generate high powered SC between approximately 1060 nm and 1800 nm. For this embodiment, the seed laser 2101 may be a 1064 nm distributed feedback (DFB) laser diode, the pre-amplifier gain fiber 2103 may be a ytterbium-doped fiber amplifier with 10/125 microns dimensions, and the pump laser 2105 may be a 10 W 915 nm laser diode. A mode field adapter may be included in the mid-stage, in addition to the isolator 2107, band pass filter 2108, polarizer 2109 and tap 2110. The gain fiber 2111 in the power amplifier may be a 20 m length of ytterbium-doped fiber with 25/400 microns dimension. The pump 2112 for the power amplifier may be up to six pump diodes providing 30 W each near 915 nm. For this much pump power, the output power in the SC may be as high as 50 W or more.

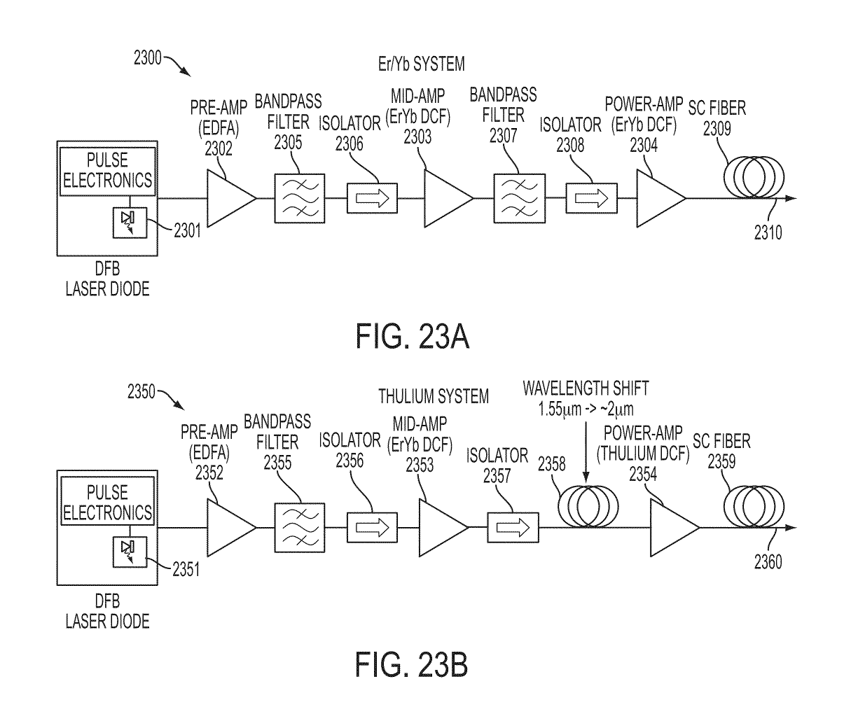

In an alternate embodiment, it may be desirous to generate high power SWIR SC over 1.4-1.8 microns and separately 2-2.5 microns (the window between 1.8 and 2 microns may be less important due to the strong water and atmospheric absorption). For example, the top SC source of FIG. 23 can lead to bandwidths ranging from about 1400 nm to 1800 nm or broader, while the lower SC source of FIG. 23 can lead to bandwidths ranging from about 1900 nm to 2500 nm or broader. Since these wavelength ranges are shorter than about 2500 nm, the SC fiber can be based on fused silica fiber. Exemplary SC fibers include standard single-mode fiber (SMF), high-nonlinearity fiber, high-NA fiber, dispersion shifted fiber, dispersion compensating fiber, and photonic crystal fibers. Non-fused-silica fibers can also be used for SC generation, including chalcogenides, fluorides, ZBLAN, tellurites, and germanium oxide fibers.

In one embodiment, the top of FIG. 23 illustrates a block diagram for an SC source 2300 capable of generating light between approximately 1400 nm and 1800 nm or broader. As an example, a pump fiber laser similar to FIG. 21 can be used as the input to a SC fiber 2309. The seed laser diode 2301 can comprise a DFB laser that generates, for example, several milliwatts of power around 1542 nm or 1553 nm. The fiber pre-amplifier 2302 can comprise an erbium-doped fiber amplifier or an erbium/ytterbium doped double clad fiber. In this example a mid-stage amplifier 2303 can be used, which can comprise an erbium/ytterbium doped double-clad fiber. A bandpass filter 2305 and isolator 2306 may be used between the pre-amplifier 2302 and mid-stage amplifier 2303. The power amplifier stage 2304 can comprise a larger core size erbium/ytterbium doped double-clad fiber, and another bandpass filter 2307 and isolator 2308 can be used before the power amplifier 2304. The output of the power amplifier can be coupled to the SC fiber 2309 to generate the SC output 2310. This is just one exemplary configuration for an SC source, and other configurations or elements may be used consistent with this disclosure.

In yet another embodiment, the bottom of FIG. 23 illustrates a block diagram for an SC source 2350 capable of generating light exemplary between approximately 1900 nm and 2500 nm or broader. As an example, the seed laser diode 2351 can comprise a DFB or DBR laser that generates, for example, several milliwatts of power around 1542 nm or 1553 nm. The fiber pre-amplifier 2352 can comprise an erbium-doped fiber amplifier or an erbium/ytterbium doped double-clad fiber. In this example a mid-stage amplifier 2353 can be used, which can comprise an erbium/ytterbium doped double-clad fiber. A bandpass filter 2355 and isolator 2356 may be used between the pre-amplifier 2352 and mid-stage amplifier 2353. The power amplifier stage 2354 can comprise a thulium doped double-clad fiber, and another isolator 2357 can be used before the power amplifier 2354. Note that the output of the mid-stage amplifier 2353 can be approximately near 1550 nm, while the thulium-doped fiber amplifier 2354 can amplify wavelengths longer than approximately 1900 nm and out to about 2100 nm. Therefore, for this configuration wavelength shifting may be required between 2353 and 2354. In one embodiment, the wavelength shifting can be accomplished using a length of standard single-mode fiber 2358, which can have exemplarly lengths between approximately 5 meters and 50 meters. The output of the power amplifier 2354 can be coupled to the SC fiber 2359 to generate the SC output 2360. This is just one exemplary configuration for an SC source, and other configurations or elements can be used consistent with this disclosure. For example, the various amplifier stages can comprise different amplifier types, such as erbium doped fibers, ytterbium doped fibers, erbium/ytterbium co-doped fibers and thulium doped fibers. One advantage of the SC lasers illustrated in FIGS. 20-23 are that they may use all-fiber components, so that the SC laser can be all-fiber, monolithically integrated with no moving parts. The all-integrated configuration can consequently be robust and reliable.

FIGS. 20-23 are examples of SC light sources that may advantageously used for SWIR light generation in various medical diagnostic and therapeutic applications. However, many other versions of the SC light sources may also be made that are intended to also be covered by this disclosure. For example, the SC generation fiber could be pumped by a mode-locked laser, a gain-switched semiconductor laser, an optically pumped semiconductor laser, a solid state laser, other fiber lasers, or a combination of these types of lasers. Also, rather than using a fiber for SC generation, either a liquid or a gas cell might be used as the nonlinear medium in which the spectrum is to be broadened.

Even within the all-fiber versions illustrated such as in FIG. 21, different configurations could be used consistent with the disclosure. In an alternate embodiment, it may be desirous to have a lower cost version of the SWIR SC laser of FIG. 21. One way to lower the cost could be to use a single stage of optical amplification, rather than two stages, which may be feasible if lower output power is required or the gain fiber is optimized. For example, the pre-amplifier stage 2102 might be removed, along with at least some of the mid-stage elements. In yet another embodiment, the gain fiber could be double passed to emulate a two stage amplifier. In this example, the pre-amplifier stage 2102 might be removed, and perhaps also some of the mid-stage elements. A mirror or fiber grating reflector could be placed after the power amplifier stage 2106 that may preferentially reflect light near the wavelength of the seed laser 2101. If the mirror or fiber grating reflector can transmit the pump light near 940 nm, then this could also be used instead of the pump combiner 2113 to bring in the pump light 2112. The SC fiber 2115 could be placed between the seed laser 2101 and the power amplifier stage 2106 (SC is only generated after the second pass through the amplifier, since the power level may be sufficiently high at that time). In addition, an output coupler may be placed between the seed laser diode 2101 and the SC fiber, which now may be in front of the power amplifier 2106. In a particular embodiment, the output coupler could be a power coupler or divider, a dichroic coupler (e.g., passing seed laser wavelength but outputting the SC wavelengths), or a wavelength division multiplexer coupler. This is just one further example, but a myriad of other combinations of components and architectures could also be used for SC light sources to generate SWIR light that are intended to be covered by this disclosure.

### Fiber Lasers Based on Cascaded Raman Shifting

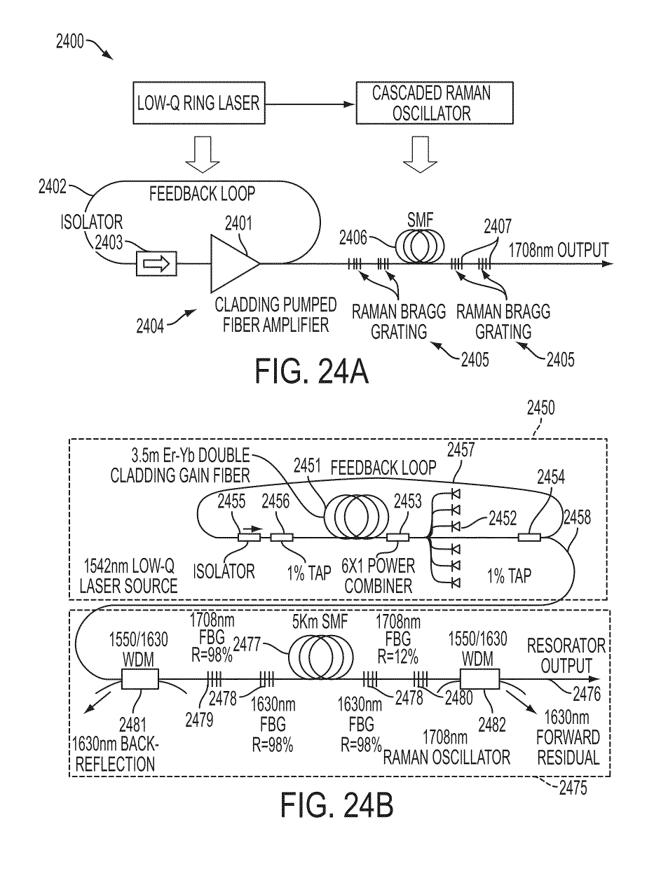

For therapeutic applications, it may be desirable to generate laser power with high spectral density in a narrower wavelength range. As an alternative to multiplexed laser diodes such as in FIG. 19, one option may be to use fiber lasers based on the cascaded Raman wavelength shifting. FIG. 24A illustrates a block diagram of one embodiment of an infrared fiber laser 2400 operating near 1720 nm. One advantage of such a configuration can be that all of the fiber parts can be spliced together to result in an all-fiber, monolithically integrated, no moving parts light source. In this particular example, the pump fiber laser 2404 can be a cladding pumped fiber amplifier 2401 with a feedback loop 2402 around the amplifier to cause lasing. In one non-limiting example, an isolator 2403 can be placed in the ring cavity of the pump laser to cause the lasing to be unidirectional. In this case, the cladding pumped fiber amplifier 2401 can be an erbium/ytterbium doped amplifier operating near 1550 nm. The pump laser light can then be coupled to a cascaded Raman oscillator 2405, where the fiber 2406 can be a single-mode fiber and two sets of Bragg gratings 2407 can be used to wavelength shift out to near 1720 nm.

In one embodiment, a specific example of the infrared fiber laser operating at approximately 1708 nm is shown in detail in FIG. 24B. The top part of the figure illustrates one embodiment of the pump fiber laser 2450 details, while the bottom part of the figure illustrates one embodiment of the cascaded Raman oscillator 2475 details. In the pump fiber laser, the gain fiber 2451 can be an erbium-ytterbium doped, double clad fiber, for example. In one embodiment, the length of the gain fiber can be between about 3 meters and 6 meters. One or more pump laser diodes 2452 can be used to excite the gain fiber 2451. In one embodiment, the pump lasers 2452 can operate at wavelengths between approximately 935 nm and 980 nm, and between 4 and 18 pump laser diodes may be used. The one or more pump laser diodes 2452 can be combined using a power combiner 2453, and then the combined pump laser diode power can be coupled to the gain fiber 2451. In this particular example, the pump laser diodes 2452 can be coupled into the gain fiber 2451 in a counter-propagating direction to the signal in the oscillator. However, the pump laser diodes could also co-propagate with the direction of the signal in the oscillator. After the pump combiner 2453, a part of the output of the gain fiber can be separated at a power tap 2454 and then fed back to the input using a feedback loop fiber 2457. In the loop, an isolator 2455 can also be inserted to permit unidirectional operation and lasing (in this particular example, the pump fiber laser 2450 resonates in a counter-clockwise direction). Other elements may also be inserted into the ring cavity, such as additional taps 2456. Although one particular example of a pump fiber laser 2450 is described, any number of changes in elements or their positions can be made consistent with this disclosure.

The bottom of FIG. 24B illustrates one embodiment of a cascaded Raman oscillator 2475 for shifting the pump fiber laser output wavelength to a longer signal wavelength 2476. The center of the oscillator is a Raman gain fiber 2477, which in this particular embodiment can be a standard single mode fiber (SMF). The length of the SMF can be in the range of about 300 m to 10 km, and as an example in this embodiment may be closer to approximately 5 km. Any number of fiber types, including high nonlinearity fibers, mid-infrared fibers, high numerical aperture fibers, or photonic crystal fibers, can be used consistent with this disclosure. The Raman gain fiber 2477 can be surrounded by a plurality of fiber Bragg gratings (FBG), 2478, 2479 and 2480. In this particular embodiment, two cascaded Raman orders are used to transfer the pump output wavelength 2458 near 1550 nm to the longer signal wavelength near 1708 nm. Hence, in FIG. 24B there can be two sets of fiber Bragg gratings (FBR).

As an example, the inner grating set 2478 can be designed to provide high reflectivity near 1630 nm. The reflectivity can be in the range of about 70% t to 90%, but in this particular embodiment can be closer to 98%. The outer grating set 2479 and 2480 can be designed to reflect light near 1708 nm (i.e., the desired longer signal wavelength). The first fiber Bragg grating 2479 can have high reflectivity, for example in the range of 70 to 90 percent, but more preferably is closer to 98%. The second fiber Bragg grating 2480 also serves as the output coupler, and hence should have a lower reflectivity value. As an example, the reflectivity of grating 2480 can be in the range of 8% to 50%, and is preferably closer to 12%.

Moreover, to remove the residual shifted pump light from the first or intermediate orders of Raman shifting, WDM couplers can be used surrounding the oscillator, such as 2481 and 2482. In this particular embodiment, the WDM couplers 2481 and 2482 are 1550/1630 couplers (i.e., couplers that pass light near 1550 nm but that couple across or out wavelengths near 1630 nm). Such couplers can help to avoid feedback into the pump fiber laser 2450 as well as minimize the residual intermediate orders in the longer signal wavelength 2476. It may also be beneficial to add an isolator between the pump fiber laser 2450 and the cascaded Raman oscillator 2475 to minimize the effects of feedback. Although one specific example is provided for the cascaded Raman oscillator 2475, any number of changes in the components or values or additional components can be made and are intended to be covered in this disclosure.

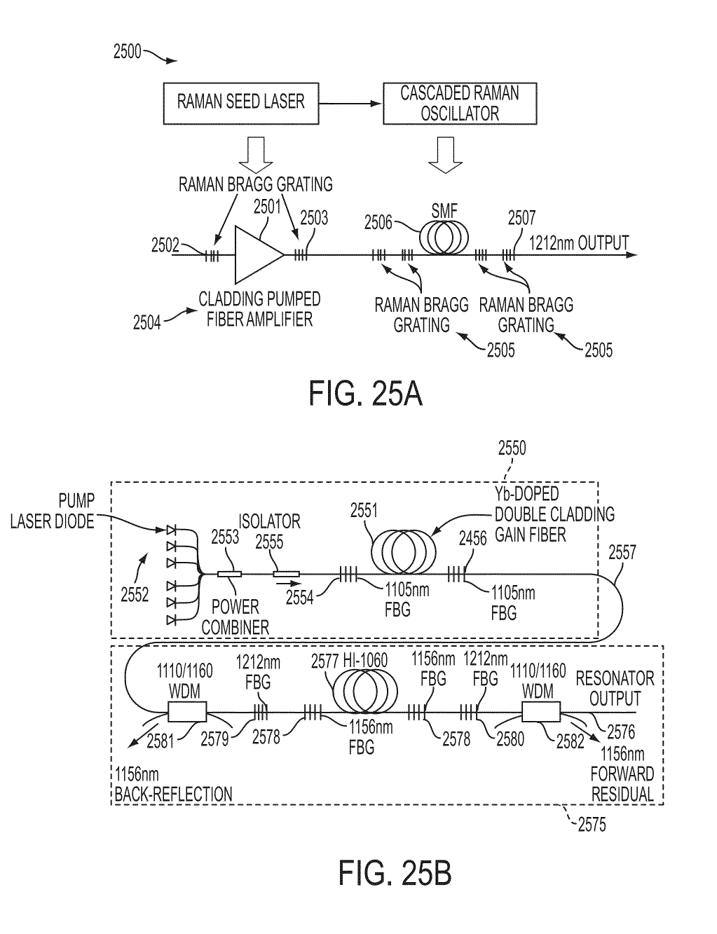

FIG. 25A illustrates a block diagram of yet another embodiment of an infrared fiber laser 2500 that operates near 1212 nm. Whereas FIG. 24 uses a ring cavity pump fiber laser, FIG. 25 uses a linear cavity pump fiber laser. Either of these configurations or other versions of the pump fiber laser can be used consistent with this disclosure. In this particular example, the pump fiber laser 2504 can be a cladding pumped fiber amplifier 2501 surrounded by fiber Bragg gratings 2502 and 2503 around the amplifier to cause lasing. In this case, the cladding pumped fiber amplifier 2501 can be a ytterbium doped amplifier operating approximately in the wavelength range between 1050 nm and 1120 nm. The pump laser light can then be coupled to a cascaded Raman oscillator 2505, where the fiber 2506 can be a single-mode fiber and two sets of Bragg gratings 2507 are used to wavelength shift out to near 1212 nm.

In yet another embodiment, a specific example of the infrared fiber laser operating at approximately 1212 nm is shown in detail in FIG. 25B. The top part of the figure illustrates one embodiment of the pump fiber laser 2550 details, while the bottom part of the figure illustrates one embodiment of the cascaded Raman oscillator 2575 details. In the pump fiber laser, the gain fiber 2551 can be a ytterbium doped, double clad fiber, for example. In one embodiment, the length of the gain fiber can be between about 3 meters and 10 meters. One or more pump laser diodes 2552 can be used to excite the gain fiber 2551. In one embodiment, the pump lasers 2552 can operate at wavelengths between approximately 850 nm and 980 nm, and between 2 and 18 pump laser diodes may be used. The one or more pump laser diodes 2552 can be combined using a power combiner 2553, and then the combined pump laser diode power can be coupled to the gain fiber 2551. After the pump combiner 2553, it may be beneficial to use one or more isolators 2555 to avoid feedback into the pump laser diodes 2552.

The pump fiber laser can be formed by using a set of gratings 2554 and 2556 around the gain fiber 2551. In one embodiment, the fiber Bragg gratings 2554 and 2556 can have reflecting at a wavelength near 1105 nm. The reflectivity of 2554 can be in the range of 70% to 90%, and in this particular embodiment can be closer to 98%. The second fiber Bragg grating 2556 can also serve as the output coupler, and hence may have a lower reflectivity value. As an example, the reflectivity of grating 2556 can be in the range of 5% to 50%, but is preferably closer to 10% in this embodiment. Other elements may also be inserted into the linear resonator cavity, such as additional taps. Although one particular example of a pump fiber laser 2550 is described, any number of changes in elements or their positions can be made consistent with this disclosure.

The bottom of FIG. 25B illustrates one embodiment of a cascaded Raman oscillator 2575 for shifting the pump fiber laser output wavelength to a longer signal wavelength 2576. The center of the oscillator is a Raman gain fiber 2577, which in this particular embodiment can be a HI-1060 fiber, which operates at a single spatial mode at the wavelengths of the ytterbium amplifier. The length of the Raman gain fiber 2577 can be in the range of 300 m to 10 km, and as an example in this embodiment may be closer to approximately 1 km. Any number of fiber types, including high nonlinearity fibers, mid-infrared fibers, high numerical aperture fibers, or photonic crystal fibers, can be used consistent with this disclosure. The Raman gain fiber 2577 can be surrounded by a plurality of fiber Bragg gratings FBG, 2578, 2579 and 2580. In this particular embodiment, two cascaded Raman orders are used to transfer the pump output wavelength 2557 near 1105 nm to the longer signal wavelength near 1212 nm. Hence, in FIG. 25B there can be two sets of fiber Bragg gratings.

As an example, the inner grating set 2578 can be designed to provide high reflectivity near 1156 nm. The reflectivity can be in the range of 70% to 90%, and in this particular embodiment can be closer to 99%. The outer grating set 2579 and 2580 can be designed to reflect light near 1212 nm (i.e., the desired longer signal wavelength). The first fiber Bragg grating 2579 can have high reflectivity, for example in the range of 70% to 90%, but in this embodiment is closer to 99%. The second fiber Bragg grating 2580 can also serve as the output coupler, and hence may have a lower reflectivity value. As an example, the reflectivity of grating 2580 can be in the range of 8% to 50%, but is closer to 25% in this embodiment.

Moreover, to remove the residual shifted pump light from the first or intermediate orders of Raman shifting, WDM couplers can be used surrounding the oscillator, such as 2581 and 2582. In this particular embodiment, the WDM couplers 2581 and 2582 are 1100/1160 couplers (i.e., couplers that pass light near 1100 nm but that couple across or out wavelengths near 1160 nm). Such couplers can help to avoid feedback into the pump fiber laser 2550 as well as minimize the residual intermediate orders in the longer signal wavelength 2576. It may also be beneficial to add an isolator between the pump fiber laser 2550 and the cascaded Raman oscillator 2575 to minimize the effects of feedback. Although one specific example is provided for the cascaded Raman oscillator 2575, any number of changes in the components or values or additional components can be made and are intended to be covered in this disclosure.

### Laser Beam Output Parameters

The laser beam output that may be used in the healthcare, medical or bio-technology applications can have a number of parameters, including wavelength, power, energy or fluence, spatial spot size, and pulse temporal shape and repetition rate. Some exemplary ranges for these parameters and some of the criteria for selecting the ranges are discussed herein. These are only meant to be exemplary ranges and considerations, and the particular combination used may depend on the details and goals of the desired procedure.

Whereas it may be advantageous in a diagnostic procedure to use a broadband laser such as a super-continuum source, for various therapeutic procedures the wavelength for the laser may be selected on the basis of a number of considerations, such as penetration depth or absorption in a particular type of tissue or water. In yet another embodiment, it may be advantageous to have the laser wavelength fall in the so-called eye-safe wavelength range. For instance, wavelengths longer than approximately 1400 nm can fall within the eye safe window. So, from an eye safety consideration there may be an advantage of using the wavelength window near 1720 nm rather than the window near 1210 nm. Thus, some of the considerations in selecting the laser wavelength range from selective tissue absorption, water absorption and scattering loss, penetration depth into tissue and eye safe operation.

Another parameter for the laser can be the energy, fluence, or pulse power density. The fluence is the energy per unit area, so it can have the units of Joules/cm². As an example, in dermatological applications or applications through the skin it may be advantageous to use fluences less than approximately 250 J/cm² to avoid burning or charring the epidermis layer. For example, therapeutic procedures may benefit from having fluences in the range of approximately 30 to 250 J/cm², preferably in the range of 50 to 200 J/cm². In another embodiment, it may even be advantageous to use lower fluence levels for therapeutic procedures to impart less pain to patients, for example in the range of approximately 30 J/cm² or less. These types of fluence levels may typically correspond to time averaged powers from the laser exceeding approximately 10 W, preferably in the power range of 10 W to 30 W, but perhaps as high as 50 W or more. Although particular fluence and power ranges are provided by way of example, other powers and fluences can be used consistent with this disclosure.

Although the output from a fiber laser may be from a single or multi-mode fiber, different spatial spot sizes or spatial profiles may be beneficial for different applications. For example, in some instances it may be desirable to have a series of spots or a fractionated beam with a grid of spots. In one embodiment, a bundle of fibers or a light pipe with a plurality of guiding cores may be used. In another embodiment, one or more fiber cores may be followed by a lenslet array to create a plurality of collimated or focused beams. In yet another embodiment, a delivery light pipe may be followed by a grid-like structure to divide up the beam into a plurality of spots. These are specific examples of beam shaping, and other apparatuses and methods may also be used and are consistent with this disclosure.

Also, various types of damage mechanisms are possible in biological tissue. In one embodiment, the damage may be due to multi-photon absorption, in which case the damage can be proportional to the intensity or peak power of the laser. For this embodiment, lasers that produce short pulses with high intensity may be desirable, such as the output from mode-locked lasers. Alternative laser approaches also exist, such as Q-switched lasers, cavity dumped lasers, and active or passive mode-locking. In another embodiment, the damage may be related to the optical absorption in the material. For this embodiment, the damage may be proportional to the fluence or energy of the pulses, perhaps also the time-averaged power from the laser. For this example, continuous wave, pulsed, or externally modulated lasers may be used, such as those exemplified in FIGS. 19-25. In one embodiment, laser pulses that are longer than approximately 100 nanoseconds to as long as 10 seconds or longer may be employed.

Particularly in the example when the damage may be related to the optical absorption, it may be beneficial to also consider the thermal diffusion into the surrounding tissue. As an example, the thermal diffusion time into tissue may be in the millisecond to second time range. Therefore, for pulses shorter than about several milliseconds, the heat may be generated locally and the temperature rise can be calculated based on the energy deposited. On the other hand, when longer pulses that may be several seconds long are used, there can be adequate time for thermal diffusion into the surrounding tissue. In this example, the diffusion into the surrounding tissue should be considered to properly calculate the temperature rise in the tissue. For these longer pulses, the particular spot exposed to laser energy will reach closer to thermal equilibrium with its surroundings. Moreover, another adjustable parameter for the laser pulses may be the rise and fall times of the pulses. However, these may be less important when longer pulses are used and the damage is related to the energy or fluence of the pulses.

Beyond having a pulse width, the laser output can also have a preferred repetition rate. For pulse repetition rates above around 10 MHz, where multiple pulses fall within a thermal diffusion time, the tissue response may be more related to the energy deposited or the fluence of the laser beam. The separation between pulses or a sub-group of pulses may also be selected so that the tissue sample can reach thermal equilibrium between pulses. Also, the pulse pattern may or may not be periodic. In one embodiment, there may be several pulses used per spot, where the pulse pattern is selected to obtain a desired thermal profile. The laser beam may then be moved to a new spot and then another pulse train delivered to that spot. In one embodiment, there can be several seconds of pre-cooling, the laser can be exposed on the tissue for several seconds, and then there may also be post-cooling. Although particular examples of laser duration and repetition rate are described, other values may also be used consistent with this disclosure. For example, depending on the application and mechanisms, the pulse rate could range all the way from continuous wave to 100's of Megahertz.

Although the present disclosure has been described in several embodiments, a myriad of changes, variations, alterations, transformations, and modifications may be suggested to one skilled in the art, and it is intended that the present disclosure encompass such changes, variations, alterations, transformations, and modifications as falling within the spirit and scope of the appended claims.

While exemplary embodiments are described above, it is not intended that these embodiments describe all possible forms of the disclosure. Rather, the words used in the specification are words of description rather than limitation, and it is understood that various changes may be made without departing from the spirit and scope of the disclosure. Additionally, the features of various implementing embodiments may be combined to form further embodiments of the disclosure. While various embodiments may have been described as providing advantages or being preferred over other embodiments with respect to one or more desired characteristics, as one skilled in the art is aware, one or more characteristics may be compromised to achieve desired system attributes, which depend on the specific application and implementation. These attributes include, but are not limited to: cost, strength, durability, life cycle cost, marketability, appearance, packaging, size, serviceability, weight, manufacturability, ease of assembly, etc. The embodiments described herein that are described as less desirable than other embodiments or prior art implementations with respect to one or more characteristics are not outside the scope of the disclosure and may be desirable for particular applications.

What is claimed is:

1. A therapeutic system comprising:
a light source generating an output optical beam, comprising:
a plurality of semiconductor sources generating an input optical beam;
a multiplexer configured to receive at least a portion of the input optical beam and to form an intermediate optical beam; and
one or more fibers configured to receive at least a portion of the intermediate optical beam and to form the output optical beam, wherein the output optical beam comprises one or more optical wavelengths, and wherein at least a portion of the one of more fibers is a fused silica fiber with a core diameter less than approximately 400 microns;
an interface device configured to receive a received portion of the output optical beam and to deliver a delivered portion of the output optical beam to a sample, wherein the interface device comprises one or more lenses to focus at least a part of the delivered portion of the output optical beam on the sample, and wherein the interface device further comprises a surface cooling apparatus to reduce damage to a top surface of the sample; and
wherein the part of the delivered portion of the output optical beam is at least partially absorbed in the sample to thermally damage at least a part of the sample, wherein a sample temperature in the part of the sample reaches about 65 Celsius or higher, while a cover temperature at the top surface of the sample remains less than about 65 Celsius, and wherein the output optical beam comprises a fluence less than about 250 Joules per centimeter squared.

2. The system of claim 1, wherein the damage to at least the part of the sample is a thermal coagulation or occlusion procedure, and the sample comprises a skin.

3. The system of claim 1, wherein the output optical beam comprises a pulse width less than several milliseconds, and wherein at least a portion of the one or more optical wavelengths is between approximately 900 to approximately 1150 nanometers, between approximately 1280 to approximately 1340 nanometers, or between approximately 1550 to approximately 1680 nanometers.

4. The system of claim 1, wherein at least some of the part of the delivered portion of the output optical beam penetrates into the sample a depth of 1.5 millimeters or more, and wherein the one or more lenses focus at least the part of the delivered portion of the output optical beam on the sample so that the focused output optical beam overcomes a Beer's law attenuation in the sample.

5. The system of claim 1, wherein the part of the delivered portion of the output optical beam further comprises a visible tracer beam, and the one or more lenses comprise an adjustable focal length system.

6. A therapeutic system comprising:
a light source generating an output optical beam, comprising:
one or more semiconductor sources generating an input optical beam;
one or more fibers configured to receive at least a portion of the input optical beam and to form an intermediate optical beam; and
a light guide configured to receive at least a portion of the intermediate optical beam and to form the output optical beam, wherein the output optical beam comprises one or more optical wavelengths;
an interface device configured to receive a received portion of the output optical beam and to deliver a delivered portion of the output optical beam to a sample, wherein the interface device comprises one or more lenses to focus at least a part of the delivered portion of the output optical beam on the sample, and wherein the interface device further comprises a surface cooling apparatus to reduce damage to a top surface of the sample; and
wherein at least some of the part of the delivered portion of the output optical beam is at least partially absorbed in the sample to thermally damage at least a part of the sample, and wherein a sample temperature in the part of the sample reaches about 65 Celsius or higher, while a cover temperature at the top surface of the sample remains less than about 65 Celsius.

7. The system of claim 6, wherein the light source comprises a plurality of semiconductor sources generating the input optical beam, and a multiplexer configured to receive at least a part of the input optical beam and further coupled to the one or more fibers.

8. The system of claim 6, wherein the part of the delivered portion of the output optical beam further comprises a visible tracer beam.

9. The system of claim 6, wherein at least a portion of the one or more optical wavelengths is between approximately 900 to approximately 1150 nanometers, between approximately 1280 to approximately 1340 nanometers, or between approximately 1550 to approximately 1680 nanometers.

10. The system of claim 6, wherein the one or more lenses focus at least the part of the delivered portion of the output optical beam on the sample so that the focused output optical beam overcomes a Beer's law attenuation in the sample.

11. The system of claim 6, wherein the one or more lenses comprise an adjustable focal length system.

12. The system of claim 6, wherein the damage to at least the part of the sample is a thermal coagulation or occlusion procedure.

13. The system of claim 6, wherein the sample is selected from the group consisting of a superficial vein, a varicose vein, a fungal infection, a hemorrhoid, a tissue welding site, a finger nail and a toe nail.

14. The system of claim 6, wherein the output optical beam comprises a pulse width less than several milliseconds.

15. The system of claim 6, wherein at least some of the part of the delivered portion of the output optical beam penetrates into the sample a depth of 1.5 millimeters or more.

16. The system of claim 6, wherein the output optical beam comprises a fluence less than about 250 Joules per centimeter squared.

17. A method of therapy comprising:
generating an output optical beam, comprising:
generating an input optical beam from one or more semiconductor sources;
forming an intermediate optical beam after propagating at least a portion of the input optical beam through one or more fibers; and
guiding at least a portion of the intermediate optical beam and forming the output optical beam, wherein the output optical beam comprises one or more optical wavelengths;
receiving a received portion of the output optical beam and delivering a delivered portion of the output optical beam to a sample;
focusing at least a part of the delivered portion of the output optical beam on the sample;
cooling a top surface of the sample;
absorbing at least some of the part of the delivered portion of the output optical beam in the sample; and
damaging thermally at least a part of the sample, and wherein a sample temperature in the part of the sample reaches about 65 Celsius or higher, while a cover temperature at the top surface of the sample remains less than about 65 Celsius.

18. The method of claim 17, wherein the output optical beam comprises a fluence less than approximately 250 Joules per squared centimeter, and wherein the sample comprises a skin.

19. The method of claim 17, wherein the output optical beam comprises a pulse width less than several milliseconds, and wherein at least a portion of the one or more optical wavelengths is between approximately 900 to approximately 1150 nanometers, between approximately 1280 to approximately 1340 nanometers, or between approximately 1550 to approximately 1680 nanometers.

20. The method of claim 17, wherein at least some of the part of the delivered portion of the output optical beam penetrates into the sample a depth of 1.5 millimeters or more.
